SEQUENCE LISTING (37 sequences)

SEQ ID NO: 1 - protein, 1520 aa, Escherichia coli
MNKKFKYKKSLLAAILSATLLAGCDGGGSGSSSDTPSVDSGSGTLPEVKPDPTPTPEPTPEPTPDPEPTPDPTPDPEPTPEPEPEPVPTKTGYLTLGGSQRVTGATCNGESSDGFTFTPGNTVSCVVGSTTIATFNTQSEAARSLRAVDKVSFSLEDAQELANSENKKTNAISLVTSSDSCPADAEQLCLTFSSVVDRARFEKLYKQIDLATDNFSKLVNEEVENNAATDKAPSTHTSTVVPVTTEGTKPDLNASFVSANAEQFYQYQPTEIILSEGQLVDSLGNGVAGVDYYTNSGRGVTDENGKFSFSWGETISFGIDTFELGSVRGNKSTIALTELGDEVRGANIDQLIHRYSTTGQNNTRVVPDDVRKVFAEYPNVINEIINLSLSNGATLDEGDQNVVLPNEFIEQFKTGQAKEIDTAICAKTDGCNEARWFSLTTRNVNDGQIQGVINKLWGVDTNYQSVSKFHVFHDSTNFYGSTGNARGQAVVNISNSAFPILMARNDKNYWLAFGEKRAWDKNELAYITEAPSIVQPENVTRDTATFNLPFISLGQVGEGKLMVIGNPHYNSILRCPNGYSWGGGVNSKGECTLSGDSDDMKHFMQNVLRYLSNDIWQPNTKSIMTVGTNLENVYFKKAGQVLGNSAPFAFHEDFTGITVKQLTSYGDLNPEEIPLLILNGFEYVTQWSGDPYAVPLRADTSKPKLTQQDVTDLIAYLNKGGSVLIMENVMSNLKEESASSFVRLLDAAGLSMALNKSVVNNDPQGYPDRVRQRRATGIWVYERYPAADGAQPPYTIDPNTGEVTWKYQQDNKPDDKPKLEVASWQEEVEGKQVTRYAFIDEAEYTTEESLEAAKAKIFEKFPGLQECKDSTYHYEINCLERRPGTDVPVTGGMYVPRYTQLNLDADTAKAMVQAADLGTNIQRLYQHELYFRTKGSKGERLNSVDLERLYQNMSVWLWNDTKYRYEEGKEDELGFKTFTEFLNCYANDAYAGGTKCSADLKKSLVDNNMIYGDGSSKAGMMNPSYPLNYMEKPLTRLMLGRSWWDLNIKVDVEKYPGSVSAKGESVTENISLYSNPTKWFAGNMQSTGLWAPAQQDVTIKSSASVPVTVTVALADDLTGREKHEVALNRPPRVTKTYTLEANGEVTFKVPYGGLIYIKGDSKDDVSANFTFTGVVKAPFYKDGEWKNDLDSPAPLGELESASFVYTTPKKNLEASNFTGGVAEFAKDLDTFASSMNDFYGRNDEDGKHRMFTYKNLTGHKHRFTNDVQISIGDAHSGYPVMNSSFSTNSTTLPTTPLNDWLIWHEVGHNAAETPLNVPGATEVANNVLALYMQDRYLGKMNRVADDITVAPEYLDESNGQAWARGGAGDRLLMYAQLKEWAEENFDIKQWYPDGELPKFYSDRKGMKGWNLFQLMHRKARGDDVGNSTFGGKNYCAESNGNAADTLMLCASWVAQADLSEFFKKWNPGASAYQLPGATEMSFQGGVSSSAYSTLASLKLPKPEKGPETINKVTEHKMSAE

SEQ ID NO: 2 - DNA, 84 bp, synthetic construct
  source 1..84: mol_type = other DNA
  misc_feature >1: note = Primer - yghJ-6xHis tagging
aaaccggaaaaagggccggaaaccattaacaaggttaccgagcataagatgtctgccgagcaccaccatcatcaccattagtaa

SEQ ID NO: 3 - DNA, 86 bp, synthetic construct
  source 1..86: mol_type = other DNA
  misc_feature >1: note = Primer - yghJ-6xHis tagging
ttgacccgatgcgccttatatcatgccggatgcggcgtgaacgccttatccggcctacaggccatatgaatatcctccttagttcc

SEQ ID NO: 4 - DNA, 23 bp, synthetic construct
  source 1..23: mol_type = other DNA
  misc_feature >1: note = Primer - yghJ-3xFLAG-His tagging, fragment A
caaacagtggtatccagatggtg

SEQ ID NO: 5 - DNA, 24 bp, synthetic construct
  source 1..24: mol_type = other DNA
  misc_feature >1: note = Primer yghJ-3xFLAG-His tagging, fragment A
ttatcgtcgtcatctttgtagtcg

SEQ ID NO: 6 - DNA, 90 bp, synthetic construct
  source 1..90: mol_type = other DNA
  misc_feature >1: note = Primer - yghJ-3xFLAG-6xHis tagging, fragment B + primer GPV187
gactacaaagaccatgacggtgattataaagatcatgatatcgactacaaagatgacgacgataaacaccaccatcatcaccattagtaa

SEQ ID NO: 7 - DNA, 108 bp, synthetic construct
  source 1..108: mol_type = other DNA
  misc_feature >1: note = Primer - yghJ-3xFLAG-10xHis tagging, fragment B + primer GPV187
gactacaaagaccatgacggtgattataaagatcatgatatcgactacaaagatgacgacgataaacaccaccatcatcaccaccatcatcaccattagtaatgtagg

SEQ ID NO: 8 - DNA, 19 bp, synthetic construct
  source 1..19: mol_type = other DNA
  misc_feature >1: note = primer - yghJ-3xFLAG-His tagging, fragment C
taggccggataaggcgttc

SEQ ID NO: 9 - DNA, 23 bp, synthetic construct
  source 1..23: mol_type = other DNA
  misc_feature >1: note = primer - yghJ-3xFLAG-His tagging, fragment C
gcttatttttgactgcgtactcg

SEQ ID NO: 10 - DNA, 91 bp, synthetic construct
  source 1..91: mol_type = other DNA
  misc_feature >1: note = primer - c4349-4351 deletion
ttgttctacaacctgtcgcccgtcaatcagcgtacggtaattgcgaccttgttgaactacttctcctgatgtgtaggctggagctgcttcg

SEQ ID NO: 11 - DNA, 94 bp, synthetic construct
  source 1..94: mol_type = other DNA
  misc_feature >1: note = primer - c4349-4351 deletion
gcatatcacgccaaccaatcaactgtaagccacgcagcgtataggtcggtgccgtggtgttgttattcatcatatgaatatcctccttagttcc

SEQ ID NO: 12 - DNA, 91 bp, synthetic construct
  source 1..91: mol_type = other DNA
  misc_feature >1: note = primer - C2308-2312 deletion
tacctctattaatcaaactgagagccgcttatttcacagcatgctctgaagtaatatggaataaattaaggtgtaggctggagctgcttcg

SEQ ID NO: 13 - DNA, 94 bp, synthetic construct
  source 1..94: mol_type = other DNA
  misc_feature >1: note = primer - C2308-2312 deletion
aatatatatatcattataaattgtcacctacccaaataaagccatgagttattaacttgcattttgaattcatatgaatatcctccttagttcc

SEQ ID NO: 14 - DNA, 91 bp, synthetic construct
  source 1..91: mol_type = other DNA
  misc_feature >1: note = primer - C2303-2307 deletion
atattaataaataaaattcaaaatgcaagttaataactcatggctttatttgggtaggtgacaatttatagtgtaggctggagctgcttcg

SEQ ID NO: 15 - DNA, 94 bp, synthetic construct
  source 1..94: mol_type = other DNA
  misc_feature >1: note = Primer - C2303-2307 deletion
ttgcaataaaaaaactgtaattttattttcactttgaaaaacctgttcttttttaacttttcggtttcatcatatgaatatcctccttagttcc

SEQ ID NO: 16 - DNA, 41 bp, synthetic construct
  source 1..41: mol_type = other DNA
  misc_feature >1: note = primer - CRISPR/Cas part 1 PCR
ccaggtctcagctctgctgaatggaagcttggattctcacc

SEQ ID NO: 17 - DNA, 45 bp, synthetic construct
  source 1..45: mol_type = other DNA
  misc_feature >1: note = primer - CRISPR/Cas part 1 PCR
ccaggtctcacgcttaagatctgactccataacagagtactcgcc

SEQ ID NO: 18 - DNA, 45 bp, synthetic construct
  source 1..45: mol_type = other DNA
  misc_feature >1: note = primer - CRISPR/Cas part 2 PCR
ccaggtctcagttttagagctagaaatagcaagttaaaataaggc

SEQ ID NO: 19 - DNA, 35 bp, synthetic construct
  source 1..35: mol_type = other DNA
  misc_feature >1: note = primer - CRISPR/Cas part 2 PCR
ccaggtctcagcaccacaggcccatggattcttcg

SEQ ID NO: 20 - DNA, 24 bp, synthetic construct
  source 1..24: mol_type = other DNA
  misc_feature >1: note = primer - waaL N20 guide sequence
agcgcgtctggcaatgtatgaagt

SEQ ID NO: 21 - DNA, 24 bp, synthetic construct
  source 1..24: mol_type = other DNA
  misc_feature >1: note = primer - waaL N20 guide sequence
aaacacttcatacattgccagacg

SEQ ID NO: 22 - DNA, 37 bp, synthetic construct
  source 1..37: mol_type = other DNA
  misc_feature >1: note = primer - delta-waaL donor DNA, up fragment
ccaggtctcagtgctggaatgttctacttaatggcga

SEQ ID NO: 23 - DNA, 46 bp, synthetic construct
  source 1..46: mol_type = other DNA
  misc_feature >1: note = primer - deltawaaL donor DNA, up fragment
agtcggaaccggtttagaaaagagtgcaacatcatcccacaaagac

SEQ ID NO: 24 - DNA, 46 bp, synthetic construct
  source 1..46: mol_type = other DNA
  misc_feature >1: note = primer - delta-waaL donor DNA, down fragment
gtctttgtgggatgatgttgcactcttttctaaaccggttccgact

SEQ ID NO: 25 - DNA, 39 bp, synthetic construct
  source 1..39: mol_type = other DNA
  misc_feature >1: note = primer - detla-waaL donor DNA, down fragment
ccaggtctcagagcatagacccaatatcacggtttatgg

SEQ ID NO: 26 - DNA, 24 bp, synthetic construct
  source 1..24: mol_type = other DNA
  misc_feature >1: note = primer - fimH N20 guide sequence
agcgagggtagtccggcagagtaa

SEQ ID NO: 27 - DNA, 24 bp, synthetic construct
  source 1..24: mol_type = other DNA
  misc_feature >1: note = primer - fimH N20 guide sequence
aaacttactctgccggactaccct

SEQ ID NO: 28 - DNA, 32 bp, synthetic construct
  source 1..32: mol_type = other DNA
  misc_feature >1: note = primer - detla-fimH donor DNA, up fragment
ccaggtctcagtgcccgcgcaaaacatccagt

SEQ ID NO: 29 - DNA, 43 bp, synthetic construct
  source 1..43: mol_type = other DNA
  misc_feature >1: note = primer - detla-fimH donor DNA, up fragment
acaaaagtcacgccaataatcgaatttaccgaccagcccatca

SEQ ID NO: 30 - DNA, 43 bp, synthetic construct
  source 1..43: mol_type = other DNA
  misc_feature >1: note = primer - detla-fimH donor DNA, down fragment
tgatgggctggtcggtaaattcgattattggcgtgacttttgt

SEQ ID NO: 31 - DNA, 34 bp, synthetic construct
  source 1..34: mol_type = other DNA
  misc_feature >1: note = primer - delta-fimH donor DNA, down fragment
ccaggtctcagagctccgtggttgggctgattat

SEQ ID NO: 32 - DNA, 24 bp, synthetic construct
  source 1..24: mol_type = other DNA
  misc_feature >1: note = primer - papG N20 guide sequence
agcggataattactatcttaccaa

SEQ ID NO: 33 - DNA, 24 bp, synthetic construct
  source 1..24: mol_type = other DNA
  misc_feature >1: note = primer - papG N20 guide sequence
aaacttggtaagatagtaattatc

SEQ ID NO: 34 - DNA, 36 bp, synthetic construct
  source 1..36: mol_type = other DNA
  misc_feature >1: note = primer - detla-papG donor DNA, up fragment
ccaggtctcagtgcactcattttggtatagcgctgt

SEQ ID NO: 35 - DNA, 41 bp, synthetic construct
  source 1..41: mol_type = other DNA
  misc_feature >1: note = primer - delta-papG donor DNA, up fragment
gaaagagtctgagccgcataagcatcattacagcctgacag

SEQ ID NO: 36 - DNA, 41 bp, synthetic construct
  source 1..41: mol_type = other DNA
  misc_feature >1: note = primer - detla-papG donor DNA, down fragment
ctgtcaggctgtaatgatgcttatgcggctcagactctttc

SEQ ID NO: 37 - DNA, 34 bp, synthetic construct
  source 1..34: mol_type = other DNA
  misc_feature >1: note = primer - delta-papG donor DNA, down fragment
ccaggtctcagagccacaacacactatccggctc

(19) United States
(12) Patent Application Publication
(10) Pub. No.: US 2026/0285915 A1
(43) Pub. Date: Sep. 24, 2026

(54) GLYCOSYLATED YGHJ POLYPEPTIDES FROM UROPATHOGENIC E. COLI

(71) Applicant: GLYPROVAC APS, Odense SØ (DK)

(72) Inventors: Anders BOYSEN, Odense SØ (DK); Mette THORSING, Tommerup (DK); Ann ZAHLE ANDERSEN, Odense S (DK)

(21) Appl. No.: 19/117,572

(22) PCT Filed: Oct. 18, 2023
(86) PCT No.: PCT/EP2023/078991

§ 371 Date: Apr. 1, 2025

(30) Foreign Application Priority Data
Oct. 19, 2022 (EP) 22202387.1

(51) Int. Cl. C07K 14/245; C12N 15/70

(52) U.S. Cl. CPC C07K 14/245; C12N 15/70

(57) ABSTRACT

The present invention relates to YGHJ polypeptides with novel glycosylation patterns. The glycosylated polypeptides are expressed in an avirulent genetically altered strain of the natural pathogenic bacteria, which gives rise to glycosylated YGHJ polypeptides which resembles the wildtype polypeptide to a higher degree than polypeptides expressed in E. coli production strains. The invention also relates to uses of such polypeptides and production strains.

### TECHNICAL FIELD OF THE INVENTION

The present invention relates to glycosylated YGHJ polypeptides from or derived from Uropathogenic Escherichia coli (UPEC) that are immunogenic. In particular, the present invention relates to compositions or vaccines comprising the glycosylated polypeptides and their application in immunization, vaccination and treatment. The invention also relates to modified production strains for such polypeptides.

### BACKGROUND OF THE INVENTION

Complex urinary tract infections (UTI) are serious conditions associated with a significant burden of morbidity and mortality in patient risk groups such as patients with diabetes, kidney stones, spinal cord dysfunction as well as all surgical patients. Risk of developing complex UTIs increase dramatically with age above 60 years (Zhao et al., 2020). Complex UTIs are often healthcare associated UTIs (HAUTI), where the UTI is a consequence of the intervention of another disease or condition. Risk of developing a hospital acquired urinary tract infection upon hospital submission is reported to range from 1.7% to as much as 4.8% (Mitchell et al., 2016).

This group of UTIs are associated with a very large risk of developing into more serious and costly infections with a potential lethal outcome. The US centre for disease control (CDC) reported that in 2002, urinary tract infections made up the highest number of hospital-acquired infections (HAIs) (>560,000) compared to other HAIs, and with a mortality rate of 2.3% was attributable to 13,000 deaths.

In addition, HAUTI's are associated with a mean increased length of hospitalization of 4 days (Mitchell et al., 2016) and direct average cost of € 5,700 per case (Cassini et al., 2016; Vallejo-Torres et al., 2018). Escherichia coli (E. coli) is the most prevalent bacteria causing HAUTI, with UPEC responsible for more than one third of HAUTI's (26%-47%) (Cek et al., 2014; The European Centre for Disease Prevention and Control, 2017; Medina and Castillo-Pino, 2019; Zhao et al., 2020).

A conservative assessment of today's UPEC HAUTI burden is >25,000 deaths and staggering € 9.5 bil. per year in direct cost in European and North American hospitals alone. Despite an enormous unmet need, no vaccines against UPEC UTI have yet successfully reached patients.

WO2006/089264 A2 discloses various open reading frames from a strain of Escherichia coli responsible for neonatal meningitis (MNEC), and a subset of these that is of particular interest for preparing compositions for immunising against MNEC infections.

WO 2011/007257 A1 discloses detoxified Escherichia coli immunogens.

Nesta et al. (PLOS Pathogens; May 2014|Volume 10|Issue 51e1004124) discloses antibodies that impair in vitro mucinase activity and in vivo colonization by both intestinal and extraintestinal E. coli strains (ExPEC's).

WO 2017/059864 A1 discloses glycosylated YGHJ polypeptides from enterotoxigenic E. coli (ETEC).

Thorsing et al. (Frontiers in Cellular and Infection Microbiology. August 2021| Volume 11| Article 705468) discloses a link between O-linked glycosylation and the relative immunogenicity of bacterial proteins and further highlights the importance of this observation in considering ETEC proteins for inclusion in future broad coverage subunit vaccine candidates.

Tapader et al. (Microbial Pathogenesis, vol. 105, 16 Feb. 2017, pages 96-99) discloses the pathogenic potential of YghJ in sepsis pathophysiology but also indicates the enterotoxic ability of YghJ.

Hence, an improved treatment of uropathogenic E. coli (UPEC) would be advantageous, and in particular a more efficient and/or reliable vaccine against uropathogenic E. coli (UPEC) would be advantageous.

### SUMMARY OF THE INVENTION

The present invention is based on the identification that YGHJ polypeptides according to the invention are more potent as vaccines when expressed in their natural host compared to when being expressed in a different expression host.

Further, the inventing team has identified unique glycosylation pattern in full length YGHJ from UPEC (see e.g. example 2), which is considered responsible for the ability to raise a high immune response when used as a vaccine and give rise to a very strong protection against infections in the bladder, as documented in the pig study illustrated in example 4.

From a production point of view, it is generally not considered desirable to express immunogens in the natural pathogenic host due to the risk of infections for the personal handling the purification process, waste management and post-production instrument handling. Therefore, polypeptide vaccines against bacterial pathogens are conventionally expressed in non-pathogenic product strains which do not require high biosafety level production facilities.

To overcome this problem, the inventing team has also developed a detoxified and non-pathogenic production strain, which is able to produce polypeptides which have glycosylation patterns according to the natural pathogenic bacteria (see example 3).

The inventing team has also identified an optimized tag for purification purposes (see example 5)

Further, the inventing team has identified that the polypeptides and compositions according to the invention are able to generate antibodies with a higher avidity compared to YGHJ versions isolated from a conventional E. coli expression strain (example 6).

Further, the inventing team has identified that expression from the production strain according to the invention resembles expression from chromosome of a wild type pathogen to a higher degree than expression from a standard E. coli production strain, such as in relation to glycan:protein ratio (example 7).

Thus, improved vaccines and production strains for such vaccines are disclosed in here.

In WO2006/089264 proteins are cloned, expressed in bacteria (non-pathogenic laboratory E. coli host, or in a Bacillus such as B. subtilis or B. megaterium). Thus, WO2006/089264 is silent in respect of the peptides being expressed in their pathogenic native host.

In WO 2011/007257 A1 it is stated that expression of peptides may take place in an E. coli strain, however preferably from a heterologous host for expression. The heterologous host may be prokaryotic (e.g. a bacterium) or eukaryotic. Suitable hosts include, but are not limited to, Bacillus subtilis, Vibrio cholerae, Salmonella typhi, Salmonella typhimurium, Neisseria lactamica, Neisseria cinerea, Mycobacteria (e.g. M. tuberculosis), yeasts, etc. (see Page 13 in WO 2011/007257 A1. Thus, WO 2011/007257 A1 is silent in respect of the peptides being expressed in their pathogenic native host. From the data presented in WO 2011/007257 A1 it appears as all peptides are expressed in E. coli BL21(DE3).

In Nesta et al. (overlapping data WO 2011/007257 A1) E. coli BL21(DE3) (Invitrogen) was also used for expression of His-tagged fusion proteins. Thus, Nesta et al. are silent in respect of the peptides being expressed in their recombinant non-pathogenic native host.

WO 2017/059864 A1 is silent in respect of the peptides being expressed in a pathogenic native UPEC host.

Thus, an object of the present invention relates to the provision of improved vaccines against UPEC.

Another object of the present invention is to provide a bacterial production strain that solves the above-mentioned problems of the prior art in relation to maintaining correct glycosylation patterns.

Thus, one aspect of the invention relates to a polypeptide comprising:
a) an amino acid sequence according to SEQ ID NO: 1; and/or
b) an amino acid sequence having at least 80% sequence identity to SEQ ID NO: 1; and/or
c) an amino acid sequence which is a fragment of at least 1000 consecutive amino acids from SEQ ID NO: 1; and/or
d) an amino acid sequence having at least 80% sequence identity to SEQ ID NO: 1 and including a fragment of at least 1000 consecutive amino acids from SEQ ID NO: 1;
   wherein said polypeptide has a glycosylation pattern defined by being glycosylated on at least 15 positions in SEQ ID NO: 1, selected from the group depicted in Table 2, such as at least 20 positions or such as at least 40 positions.

Another aspect of the present invention relates to a composition comprising the polypeptide according to the invention and/or a composition comprising a plurality of polypeptides with a glycosylation pattern according to the invention.

Yet another aspect of the present invention is to provide the polypeptide according to the invention or the composition according to the invention for use as a medicament, such as a vaccine.

A further aspect relates to the polypeptide according to the invention or the composition according to the invention for use in the treatment, prevention and or alleviation of E. coli infections, preferably urinary tract infections (UTIs), such as bladder infection.

Yet an aspect relates to a genetically modified E. coli which does not express:
FimH;
PapG; and
WaaL.

Still another aspect of the present invention is to a process for producing a glycosylated polypeptide of interest, the process comprising
a) expressing the polypeptide of interest in the genetically modified E. coli according to the invention; and
b) purifying the glycosylated polypeptide of interest from said bacteria.

An aspect also relates to a glycosylated polypeptide obtained by or obtainable by a process according to the invention.

Also, the invention relates to an antibody specific for the polypeptide according to the invention, such as a monoclonal or polyclonal antibody.

### BRIEF DESCRIPTION OF THE FIGURES

FIG. 1 Number of identified glycosylated peptides as function of input sample amount (μg). The total number of glycopeptides derived from hyperglycosylated full length YGHJ (GPV02) isolated from the production stain (circles) or full length YGHJ isolated from an E. coli over-expression strain (square) as a function of sample input is plotted. The black dots show the number of glycopeptides identified in a total of 12 BEMAP analyses using GPV02 (full length YGHJ isolated from the Production strain as defined in this invention). The black square shows the number of glycopeptides identified in 1 BEMAP analysis of full length YGHJ isolated from a conventional E. coli over-expression strain.

FIG. 2 The relative abundance of glycopeptides derived from GPV02 isolated from either the production stain as defined in this invention or full length YGHJ isolated from a conventional E. coli over-expression strain is plotted. Triangles show the relative abundance of four BEMAP analyses from GPV02 isolated from the production strain. Black squares show the relative abundance of full length YGHJ peptides isolated from an E. coli over-expression strain. Peptides not identified in either of the five studies are shown with an abundance of 0.0001%.

FIG. 3 Schematic of the lipopolysaccharide (LPS) biosynthesis in E. coli in which gene products involved in the synthesis are shown. These were targeted for deletion to obtain a strain producing O-antigen-free LPS. O-antigen subunits are synthesized on the inner side of the cytoplasmic membrane on an undecaprenyl diphosphate carrier and flipped to the outer side by Wzx. Here, subunits are polymerized to long chains by Wzy and Wzz and ligated to a Lipid A-core molecule by WaaL to form a complete LPS molecule. The free undecaprenyl carrier is recycled to the inner face of the cytoplasmic membrane and reused in another step. The entire LPS molecule is transported across the periplasmic space and outer membrane, in which it is incorporated in the outer leaflet. The figure is adapted from (Wang and Quinn, 2010).

FIG. 4 Western blot of O-antigen levels in culture supernatant from the UTI89 wildtype and its Δc4349-c4351 mutant. The strains were grown to the same optical density and bacteria free supernatant samples were collected. The samples were separated by SDS PAGE and transferred to a PVDF membrane. O-antigen was detected using primary pig antibodies against the 018 serotype and secondary HRP-conjugated antibodies against pig IgG. The antibody complexes were visualized by chemiluminescence after wetting with Immobilon Forte Western HRP substrate.

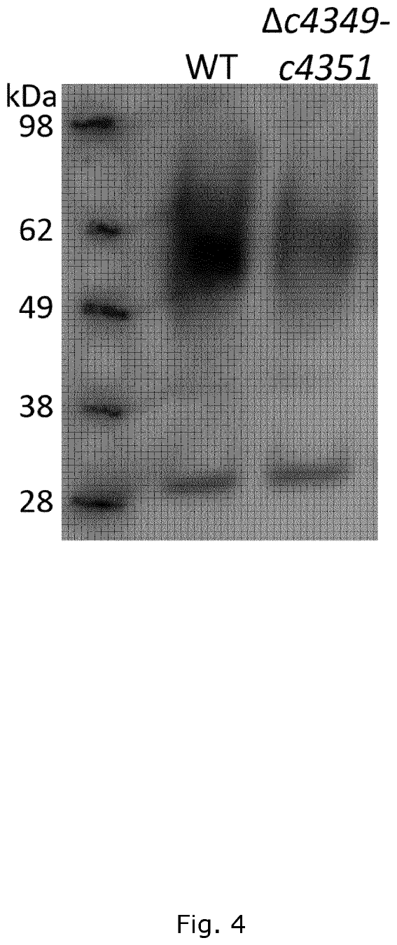

FIG. 5 Western blot of O-antigen levels in culture supernatant from the UTI89 wildtype and its ΔwaaL mutant prepared as described in FIG. 4. The wildtype sample was 2-fold serial diluted before SDS PAGE to aid the estimation of the relative signal intensity between wildtype and mutant.

FIG. 6 Western blots of full length YGHJ levels in culture supernatants (upper panel) and intracellular levels of σ^{E} (lower panel) of UTI89 wildtype and ΔwaaL ygh_::3×FLAG-10×HHis. Sample collection and analysis as described in FIG. 4; for intracellular levels, bacterial pellets were collected. YGHJ was detected with primary rabbit antibodies raised against full length YGHJ and secondary HRP-conjugated anti-rabbit antibodies, and σ^{E} was detected with primary mouse anti-σ^{E} antibodies and secondary HRP-conjugated anti-mouse antibodies.

FIG. 7 Adhesion assay showing the relative ability of the UPEC wildtype, the production strain as defined in this invention and a non-pathogenic E. coli strain to adhere to human bladder cells in vitro. The adhesive capacity is relative to the wildtype (100%). Bars represent the mean with SD of four biological replicates, each with eight technical replicates. p-value determined using a two-tailed unpaired student t test assuming Gaussian distribution.

FIG. 8 The number of colony forming units (CFU) associated with the pig bladder tissue on the last day of the challenge experiment is shown. The CFU count for the 19 vaccinated pigs is plotted as filled circles. The CFU count for the 17 control pigs is plotted as filled triangles. The geometric mean is shown. Statistical analysis: Two-tailed unpaired student t test not assuming Gaussian distribution (Mann-Whitney test). Exact P value is shown.

FIG. 9 The number of colony forming units (CFU) in the pig urine one day post infection is shown. The CFU count for the 19 vaccinated pigs is plotted as filled circles. The CFU count for the 17 control pigs is plotted as filled triangles. The geometric mean is shown. Statistical analysis: Two-tailed unpaired student t test not assuming Gaussian distribution (Mann-Whitney test). Bars indicate geometric mean values. Exact P value is shown.

FIG. 10 The serum IgG avidity for four pigs vaccinated with either glycosylated or non-glycosylated GPV02 is shown. Statistical analysis: Two-tailed paired student t test assuming Gaussian distribution. Mean values and standard deviation is plotted. Exact P value is shown.

FIG. 11 The glycan to protein ratio is shown for GPV02 isolated from either a wild type UTI89 strain, the production strain as defined in this invention or a conventional E. coli production strain. Glycan content was determined using FTIR (fourier-transform infrared spectroscopy).

The present invention will now be described in more detail in the following.

### DETAILED DESCRIPTION OF THE INVENTION

### Definitions

Prior to discussing the present invention in further details, the following terms and conventions will first be defined:

Uropathoaenic E. coli (UPEC)

Uropathogenic E. coli (UPEC) is a main cause of urinary tract infections (UTI). In ascending infections, bacteria colonize the urethra and spread up the urinary tract to the bladder as well as to the kidneys (causing pyelonephritis), or the prostate in males. Because women have a shorter urethra than men, they are more likely to suffer from an ascending UTI.

Uropathogenic E. coli (UPEC) is part of the extraintestinal pathogenic E. coli (ExPEC) pathotype.

### GPV02

In the present context the term “GPV02” refers to the hyperglycosylated full length YGHJ polypeptide according to the present invention.

Thus, in an embodiment, GPV02 is obtained by purification from the purification strain as defined in the present invention. In the example section GPV02 is obtained from the production strain according to example 3.

### Glycosylation

The term “glycosylation” refers to O-linked glycosylation. This is the attachment of a sugar molecule to a hydroxyl oxygen (hence O-linked) of either a Serine or Threonine side chain in a protein.

### Sequence Identity

The term “sequence identity” indicates a quantitative measure of the degree of homology between two amino acid sequences of substantially equal length or between two nucleic acid sequences of substantially equal length. The two sequences to be compared must be aligned to best possible fit with the insertion of gaps or alternatively, truncation at the ends of the protein sequences. The sequence identity can be calculated as

$$\frac{( {N}_{ref} - {N}_{dif} ) 100}{{N}_{ref}} ,$$

wherein N_{dif} is the total number of non-identical residues in the two sequences when aligned and wherein N_{ref} is the number of residues in one of the sequences. Hence, the DNA sequence AGTCAGTC will have a sequence identity of 75% with the sequence AATCAATC (N_{dif}=2 and N_{ref}=8). A gap is counted as non-identity of the specific residue(s), i.e., the DNA sequence AGTGTC will have a sequence identity of 75% with the DNA sequence AGTCAGTC (N_{dif}=2 and N_{ref}=8). Sequence identity can alternatively be calculated by the BLAST program e.g., the BLASTP program (W. R Pearson and D. J. Lipman (1988)). In one embodiment of the invention, alignment is performed with the sequence alignment method ClustalW with default parameters as described by J. D. Thompson et al (1994), available at http://www2.ebi.ac.uk/clustalw/.

For calculations of sequence identity when comparing polypeptide fragments with longer amino acid sequences, the polypeptide fragment is aligned with a segment of the longer amino acid sequence. The polypeptide fragment and the segment of the longer amino acid sequence may be of substantially equal length. Thus, the polypeptide fragment and the segment of the longer amino acid sequence may be of equal length. After alignment of the polypeptide fragment with the segment of the longer amino acid sequence, the sequence identity is computed as described above.

A preferred minimum percentage of sequence identity is at least 80%, such as at least 85%, at least 90%, at least 91%, at least 92%, at least 93%, at least 94%, at least 95%, at least 96%, at least 97%, at least 98%, at least 99%, and at least 99.5%.

Thus, one embodiment of the present invention relates to a polypeptide as described herein, wherein the polypeptide or polypeptide fragment has at least 80% sequence identity to the full-length sequence of SEQ ID No: 1, such as at least 80%, such as at least 85%, at least 90%, at least 91%, at least 92%, at least 93%, at least 94%, at least 95%, at least 96%, at least 97%, at least 98%, at least 99%, or at least 99.5%,

An embodiment of the present invention relates to a polypeptide as described herein, wherein the polypeptide or polypeptide fragment has at least 90% sequence identity to SEQ ID NO: 1.

### Immunogenic Polypeptide

An immunogenic polypeptide is defined as a polypeptide that induces an immune response. The immune response may be monitored by one of the following methods:

An in vitro cellular response is determined by release of a relevant cytokine such as IFN-γ, from lymphocytes withdrawn from an animal or human currently or previously infected with UPEC, or by detection of proliferation of these T cells. The induction is performed by addition of the polypeptide or the immunogenic part to a suspension comprising from 1×10⁵ cells to 3×10⁵ cells per well. The cells are isolated from either blood, the spleen, the liver or the lung and the addition of the polypeptide or the immunogenic part of the polypeptide result in a concentration of not more than 20 μg per ml suspension and the stimulation is performed from two to five days. For monitoring cell proliferation, the cells are pulsed with radioactive labeled Thymidine and after 16-22 hours of incubation, the proliferation is detected by liquid scintillation counting. A positive response is a response more than background plus two standard deviations. The release of IFN-γ can be determined by the ELISA method, which is well known to a person skilled in the art. A positive response is a response more than background plus two standard deviations. Other cytokines than IFN-γ could be relevant when monitoring an immunological response to the polypeptide, such as IL-12, TNF-α, IL-4, IL-5, IL-10, IL-6, TGF-γ.

Another and more sensitive method for determining the presence of a cytokine (e.g. IFN-γ) is the ELISPOT method where the cells isolated from either the blood, the spleen, the liver or the lung are diluted to a concentration of preferable of 1 to 4×10⁶ cells/ml and incubated for 18-22 hours in the presence of the polypeptide or the immunogenic part of the polypeptide resulting in a concentration of not more than 20 μg per ml. The cell suspensions are hereafter diluted to 1 to 2×10⁶/ml and transferred to Maxisorp plates coated with anti-IFN-γ and incubated for preferably 4 to 16 hours. The IFN-γ producing cells are determined by the use of labelled secondary anti-IFN-antibody and a relevant substrate giving rise to spots, which can be enumerated using a dissection microscope. It is also a possibility to determine the presence of mRNA coding for the relevant cytokine by the use of the PCR technique. Usually, one or more cytokines will be measured utilizing for example the PCR, ELISPOT or ELISA. It will be appreciated by a person skilled in the art that a significant increase or decrease in the amount of any of these cytokines induced by a specific polypeptide can be used in evaluation of the immunological activity of the polypeptide.

An in vitro cellular response may also be determined by the use of T cell lines derived from an immune individual or an UPEC infected person where the T cell lines have been driven with either live UPEC, extracts from the bacterial cell or culture filtrate for 10 to 20 days with the addition of IL-2. The induction is performed by addition of not more than 20 μg polypeptide per ml suspension to the T cell lines containing from 1×10⁵ cells to 3×10⁵ cells per well and incubation is performed from two to six days. The induction of IFN-γ or release of another relevant cytokine is detected by ELISA. The stimulation of T cells can also be monitored by detecting cell proliferation using radioactively labeled Thymidine as described above. For both assays, a positive response is a response more than background plus two standard deviations.

An in vitro humoral response is determined by a specific antibody response in an immune or infected individual. The presence of antibodies may be determined by an ELISA technique or a Western blot where the polypeptide or the immunogenic part is absorbed to either a nitrocellulose membrane or a polystyrene surface. The serum is preferably diluted in PBS from 1:10 to 1:100 and added to the absorbed polypeptide and the incubation being performed from 1 to 12 hours. By the use of labeled secondary antibodies the presence of specific antibodies can be determined by measuring the presence or absence of a specific label e.g. by ELISA where a positive response is a response of more than background plus two standard deviations or alternatively a visual response in a Western blot.

Another relevant parameter is measurement of the protection in animal models induced after vaccination with the polypeptide in an adjuvant or after DNA vaccination. Suitable animal models include primates, guinea pigs or mice, which are challenged with an infection of a UPEC. Readout for induced protection could be decrease or absence of the bacterial load in target organs compared to non-vaccinated animals, prolonged survival times compared to non-vaccinated animals and diminished weight loss, or pathology compared to non-vaccinated animals.

Thus, the glycosylated polypeptides described herein are immunogenic when one of the above-described tests is positive.

### Polypeptide

As described above, and outlined in the example section, the inventing team has designed an E. coli expression strain which is non-pathogenic but able to produce GPV02, a highly glycosylated YGHJ immunogenic protein which has shown to be very efficient in a pig immunization trial (see Example 4). Thus, an aspect of the invention relates to a polypeptide comprising:
a) an amino acid sequence according to SEQ ID NO: 1; and/or
b) an amino acid sequence having at least 80% sequence identity to SEQ ID NO: 1; and/or
c) an amino acid sequence which is a fragment of at least 1000 consecutive amino acids from SEQ ID NO: 1; and/or
d) an amino acid sequence having at least 80% sequence identity to SEQ ID NO: 1 and including a fragment of at least 1000 consecutive amino acids from SEQ ID NO: 1;
   wherein said polypeptide has a glycosylation pattern defined by being glycosylated on at least 15 positions in SEQ ID NO: 1, selected from the group depicted in Table 2A-B, such as at least 20 positions or such as at least 40 positions.

In an embodiment, the polypeptide is glycosylated on at least 15 positions such as on at least 20 positions, such as all of the positions depicted in Table 5B. In an embodiment, the polypeptide is glycosylated on at least 40 positions such as on at least 50 positions, such as all of the positions depicted in Table 5A-B. As shown in Example 2, Table 2 lists all the overall identified glycosylation sites.

In another embodiment, the polypeptide is glycosylated on at least 15 positions selected from the group depicted in Table 3A-B, such as on at least 20 positions, such as on at least 30 positions, or such as all of the positions according to Table 3A-B.

In yet another embodiment, the polypeptide is glycosylated on at least 15 positions selected from the group depicted in Table 3B, such as on at least 20 positions, such as on at least 30 positions, or such as all of the positions according to Table 3B. Table 3 lists 41 sites (all sites also part of Table 2), which are considered the most abundantly identified sites (see also Example 2).

In yet an embodiment, the polypeptide is glycosylated on at least 10 positions selected from the group depicted in Table 4, such as on at least 15 positions, such as on at least 20 positions or such as all of the positions depicted in Table 4. Table 4 lists (all part of Table 2 and Table 3) newly identified sites which has not previously been identified (see also Example 2).

Very abundantly identified glycosylation-sites are S152, S154 and S164. Thus, in a preferred embodiment, the polypeptide is glycosylated on at least position S152, and/or S154 and/or S164, such as S152 and S154, such as S154 and S164, such as S152 and S164 or such as S152, S154 and S164.

Other very abundantly identified glycosylation-sites are T592 and S594 and S597. Thus, in another preferred embodiment, the polypeptide is glycosylated on at least positions T592, and/or S594 and/or S597, such as T592 and S594, such as S594 and S597, such as T592 and S597, or such as T592 and S594 and S597.

In yet an embodiment, the amino acid sequence according to according to b) has at least 85% sequence identity to SEQ ID NO: 1, such as at least 90% sequence identity, such as at least 95%, such as least 99% sequence identity to SEQ ID NO: 1.

In a further embodiment, the amino acid sequence according to c) or d) is a fragment of at least 1100 consecutive amino acids from SEQ ID NO: 1, such as at least 1200 consecutive amino acids, such as at least 1300 consecutive amino acids, such as at least 1400 consecutive amino acids or such as at least 1500 consecutive amino acids from SEQ ID NO: 1. SEQ ID NO: 1 has a length of 1520 AA, thus larger glycosylated fragments are considered also to be able to give rise to an immunogenic response.

In an embodiment, the polypeptide is derived from an ExPEC such as a UPEC strain. In an embodiment, the polypeptide is derived from a production strain according to the invention.

In another embodiment, said polypeptide is immunogenic, such as being a vaccine.

In a preferred embodiment, the polypeptide is SEQ ID NO: 1.

Tags may improve purification of the polypeptide. Thus, in an embodiment, the polypeptide comprises a Flag-tag and/or a His-tag, preferably at the C-terminal.

In a preferred embodiment, the polypeptide comprises a 3×FLAG-6×His tag or a 3×FLAG-10×His tag, preferably at the C-terminal end. As shown in example 5, improved tags have been designed for the polypeptide according to the invention.

In a preferred embodiment, the invention relates to a polypeptide comprising:
a) an amino acid sequence according to SEQ ID NO: 1;
   wherein said polypeptide has a glycosylation pattern defined by being glycosylated on at least 15 positions in SEQ ID NO: 1, selected from the group depicted in Table 2B;
   and
being glycosylated on at least two of positions S152, S154 and S164; and
being glycosylated on at least two of positions T592, S594 and S597.

In another preferred embodiment, the invention relates to a polypeptide comprising:
a) an amino acid sequence according to SEQ ID NO: 1;
   wherein said polypeptide has a glycosylation pattern defined by being glycosylated on at least 10 positions selected from the group depicted in Table 4, such as on at least 15 positions, such as on at least 20 positions or such as all of the positions depicted in Table 4.

The polypeptide has a glycan to protein ratio, by weight (preferably measured by Fourier-transform Infrared spectroscopy, such as described in example 7), of at least 0.020, such as at least 0.025, such as at least 0.030, such as at least 0.034, such as in the range 0.025-0.034, such as in the range 0.020-0.030, such as 0.020-0.025.

In a further preferred embodiment, the polypeptide has a glycan to protein ratio, by weight, of at least 0.020, such as at least 0.025, preferably at least 0.030 or such as in the range 0.020-0.050, by weight, preferably in the range 0.030-0.050 more preferably in the range 0.030-0.04. As shown in example 7, a high glycan to protein ratio is important to resemble YghJ produced in its wt strain.

In an embodiment, the glycan to protein ratio is determined by Fourier-Transform Infrared Spectroscopy (FTIR). In example 7 FTIR has been used.

In yet a preferred embodiment, the polypeptide is an ExPEC-derived polypeptide, such as an UPEC-derived polypeptide.

In another preferred embodiment, said polypeptide has a glycosylation pattern defined by being glycosylated on at least 40 positions in SEQ ID NO: 1, selected from the group depicted in Table 2A-B.

### Composition

The invention also relates to compositions comprising the polypeptide according to the invention. Thus, another aspect of the invention relates to a composition comprising the polypeptide according to the invention and/or a composition comprising a plurality of polypeptides with a glycosylation pattern according to the invention.

It is to be understood that in an aspect of the invention, in a composition a single polypeptide may not comprise all of the glycosylations as defined for the polypeptide according to the invention, but the glycosylations may be distributed among different polypeptides. Due to the way the bacteria will attach the glycosylations, each unique polypeptide may not have identical glycosylation patterns, albeit the patters according to claim 1 are indeed expected.

Thus, in an embodiment, the plurality of polypeptides has an overall glycosylation pattern according to the invention. The overall glycosylation pattern may be determined using the BEMAP method described in example 2.

In an embodiment, the composition is a pharmaceutical composition.

In another embodiment, the composition further comprises a pharmaceutically acceptable carrier, diluent, and/or adjuvant.

In a preferred embodiment, the adjuvant is selected from the group consisting of dmLT, Litevax CMS and combinations thereof. These adjuvants have been used in the pig trial (Example 4—Pig challenge study).

“Litevax CMS” is an adjuvant comprising “Carbohydrate Mono Sulphate ester/Squalane/Polysorbate 80 emulsion in PBS 40 mg/mL CMS”. Thus, “Litevax CMS is a synthetic carbohydrate fatty acid monosulphate derivative (‘CMS’) immobilized on nano-droplets of a squalane-in-water emulsion. The concentration is expressed in mg of CMS and the w/w ratio of CMS/Polysorbate 80/squalane is 1:1:2. (see also (WO2016013938—ADJUVANTS) and (Hilgers et al., 2017)

The adjuvant “LT(R192G/L211A)” or “dmLT” is a detoxified version of the heat-labile enterotoxin of Escherichia coli, with two mutations in its A-subunit that remove the enterotoxicity but preserve the adjuvanticity of the molecule. DmLt is also described in U.S. Pat. No. 6,033,673.

In another embodiment, the adjuvant is selected from the group consisting of dimethyloctadecylammonium bromide (DDA), dimethyloctadecenylammonium bromide (DODAC), Quil A, poly I:C, aluminium hydroxide, Freund's incomplete adjuvant, IFN-γ, IL-2, IL-12, monophosphoryl lipid A (MPL), Treholose Dimycolate (TDM), Trehalose Dibehenate and muramyl dipeptide (MDP).

Pharmaceutical compositions comprising the polypeptides described herein may be administered in a physiologically acceptable medium (e.g., deionized water, phosphate buffered saline (PBS), saline, aqueous ethanol or other alcohol, plasma, proteinaceous solutions, mannitol, aqueous glucose, vegetable oil, or the like).

Thus, an embodiment of the present invention relates to a composition comprising a polypeptide as described herein that constitutes a pharmaceutical composition.

Buffers may also be included, particularly where the media are generally buffered at a pH in the range of about 5 to 10, where the buffer will generally range in concentration from about 50 to 250 mM salt, where the concentration of salt will generally range from about 5 to 500 mM, physiologically acceptable stabilizers, and the like.

The compounds may be lyophilized for convenient storage and transport.

Thus, in a further embodiment of the present invention the composition comprises one or more excipients, diluents and/or carriers.

Aqueous suspensions may contain the active materials in admixture with excipients suitable for the manufacture of aqueous suspensions.

Such excipients include suspending agents, for example sodium carboxymethylcellulose, methylcellulose, hydropropyl-methylcellulose, sodium alginate, polyvinylpyrrolidone, gum tragacanth and gum acacia; dispersing or wetting agents can be a naturally-occurring phosphatide, for example, lecithin, or condensation products of an alkylene oxide with fatty acids, for example polyoxyethylene stearate, or condensation products of ethylene oxide with long chain aliphatic alcohols, for example heptadecaethyleneoxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate.

The vaccines are administered in a manner compatible with the dosage formulation, and in such amount as will be prophylactic or therapeutically effective and immunogenic. The quantity to be administered depends on the subject to be treated, including, e.g., the capacity of the individual's immune system to mount an immune response, and the degree of protection desired. Suitable dosage ranges are of the order of several hundred micrograms of the polypeptide of the invention per vaccination with a preferred range from about 0.1 μg to 1000 μg, such as in the range from about 1 μg to 300 μg, and especially in the range from about 10 μg to 100 μg. Suitable regimens for initial administration and booster shots are also variable but are typified by an initial administration followed by subsequent inoculations or other administrations.

The manner of application may be varied widely. Any of the conventional methods for administration of a vaccine are applicable. These include oral, nasal or mucosal application in either a solid form containing the active ingredients (such as a pill, suppository or capsule) or in a physiologically acceptable dispersion, such as a spray, powder or liquid, or parenterally, by injection, for example, subcutaneously, intradermally or intramuscularly or transdermally applied. The dosage of the vaccine will depend on the route of administration and will vary according to the age of the person to be vaccinated and, to a lesser degree, the size of the person to be vaccinated. Currently, most vaccines are administered intramuscularly by needle injection, and this is likely to continue as the standard route. However, vaccine formulations that induce mucosal immunity have been developed, typically by oral or nasal delivery. One of the most widely studied delivery systems for induction of mucosal immunity contains cholera toxin (CT) or its B subunit. This protein enhances mucosal immune responses and induces IgA production when administered in vaccine formulations. An advantage is the ease of delivery of oral or nasal vaccines. Modified toxins from other microbial species, which have reduced toxicity but retained immunostimulatory capacity, such as modified heat-labile toxin from Gram-negative bacteria or staphylococcal enterotoxins may also be used to generate a similar effect. These molecules are particularly suited to mucosal administration.

The vaccines are conventionally administered parenterally, by injection, for example, either subcutaneously or intramuscularly. Additional formulations which are suitable for other modes of administration include suppositories and, in some cases, oral formulations. For suppositories, traditional binders and carriers may include, for example, polyalkalene glycols or triglycerides; such suppositories may be formed from mixtures containing the active ingredient in the range of 0.5% to 10%, preferably 1-2%. Oral formulations include such normally employed excipients as, for example, pharmaceutical grades of mannitol, lactose, starch, magnesium stearate, sodium saccharine, cellulose, magnesium carbonate, and the like. These compositions take the form of solutions, suspensions, tablets, pills, capsules, sustained release formulations or powders and advantageously contain 10-95% of active ingredient, preferably 25-70%.

In an embodiment, the composition is a pharmaceutical composition, such as an immunogenic composition such as a vaccine.

In yet an embodiment, the composition is formulated for intradermal, transdermal, subcutaneous, intramuscular or mucosal application, preferably for subcutaneous application.

Genetically Modified E. coli

As outlined in Example 3—Non-pathogenic production strain, the inventing team has generated a non-pathogenic UPEC strain for production of YGHJ, which retains its ability to effectively glycosylate YGHJ. Hence, an aspect of the invention relates to a genetically modified E. coli which does not express:
FimH; and/or
PapG; and/or
WaaL.

In a preferred embodiment, the genetically modified E. coli does not express FimH and WaaL. As shown in example 3, such a construct also has a reduced adhesion to bladder cells.

In an even more preferred embodiment, the genetically modified E. coli does not express:
FimH;
PapG; and
WaaL.

In an embodiment, the genes are knocked out by a method selected from the group consisting of in-frame deletions, introduction of stop sites and whole gene removal. In the example section expression was stopped (knocked out) by CRISPR/CAS and Datsenko & Wanner (Datsenko and Wanner, 2000). The skilled person will of course be able to use other methods to knock-out the same genes.

The genetically modified E. coli may express an (endogenous) protein of interest or be adapt to be transfected with a gene of interest. Thus, in an embodiment,
said genetically modified E. coli express a glycosylated polypeptide of interest, such as an immunogenic vaccine; or
is adapted to express a glycosylated polypeptide of interest, such as an immunogen/vaccine.

In the present context the term “adapted to express” is to be understood as the strain being adapted to subsequently being modified to express a gene-construct of interest which at this stage is not present in the strain; Hence the strain is “adapted to express”.

In an embodiment, the genetically modified E. coli is an ExPEC, such as UPEC, such as UTI89. In a preferred embodiment, the genetically modified E. coli is a UPEC E. coli, such as UTI89.

In an embodiment, the genetically modified E. coli encodes for a polypeptide which can be expressed in said E. coli, with a glycosylation pattern similar to a non-genetically modified version of said E. coli (such as UTI89).

In another embodiment, the genetically modified E. coli encodes for a polypeptide according to the invention.

In a related embodiment, the genetically modified E. coli encodes for a polypeptide which contains a glycosylation pattern as defined for the present invention.

In an embodiment said polypeptide is under the control of the endogenous promoter. This could be the case when it is the wild type protein which is expressed (e.g. with a tag). Thus, the protein of interest may not be located on a plasmid.

In an embodiment, said polypeptide is expressed from the genome, such as being an endogenous polypeptide and not expressed from an exogenous vector, such as a plasmid. In the example section it is wildtype YGHJ which is expressed from the genome of the bacteria.

A polypeptide of interest may however also be expressed from an exogenous vector, such as a plasmid. Thus, in an embodiment the polypeptide is expressed from an exogenous vector, such as a plasmid.

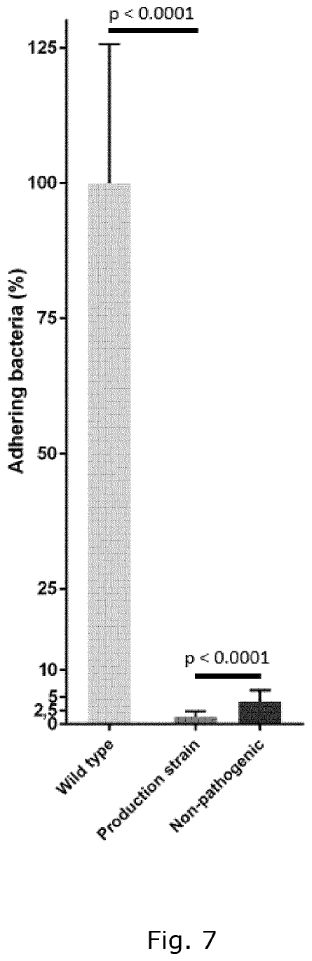

As outlined in example 3 (see also FIG. 7) the production strain is considered non-pathogenic by having e.g. very low adherence liver bladder cells. Thus, in an embodiment, the genetically modified E. coli is avirulent and/or non-pathogenic and/or not capable of causing disease.

In a related embodiment, the genetically modified E. coli has lower adherence to human bladder cells, such as human bladder cell line 5637, than a corresponding wildtype strain.

In another related embodiment, the genetically modified E. coli has lower adherence to human bladder cells, such as human bladder cell line 5637, than E. coli K-12 MG1655.

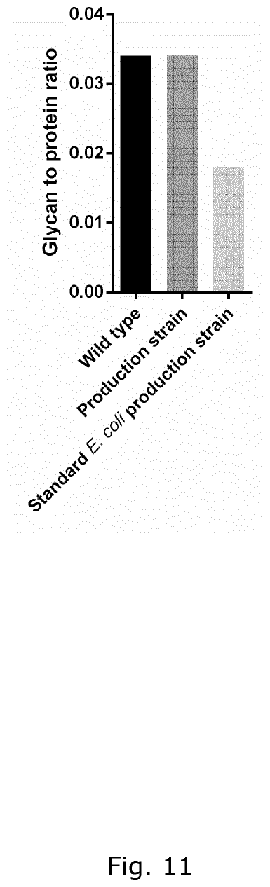

In an embodiment, the production strain ensures a glycan to protein ratio identical or similar to a wild type UPEC strain. This is shown in example 7 and FIG. 11.

In a preferred embodiment, the genetically modified E. coli, expresses an ExPEC-derived polypeptide, such as an UPEC-derived polypeptide comprising:
a) an amino acid sequence according to SEQ ID NO: 1; and/or
b) an amino acid sequence having at least 90% sequence identity to SEQ ID NO: 1; and/or
c) an amino acid sequence which is a fragment of at least 1000 consecutive amino acids from SEQ ID NO: 1; and/or
d) an amino acid sequence having at least 90% sequence identity to SEQ ID NO: 1 and including a fragment of at least 1000 consecutive amino acids from SEQ ID NO: 1;
   wherein said polypeptide has a glycosylation pattern defined by being glycosylated on at least 15 positions in SEQ ID NO: 1, selected from the group depicted in Table 2A-B, such as at least 20 positions or such as at least 40 positions.

### Process for Producing Polypeptide

In an aspect the invention relates to a process for producing a glycosylated polypeptide of interest and/or plurality of glycosylated polypeptides of interest, the process the process comprising
a) expressing the polypeptide of interest in the genetically modified E. coli according to the invention; and
b) purifying the glycosylated polypeptide of interest and/or plurality of glycosylated polypeptides of interest from said bacteria, such as from the lysate and/or medium and/or supernatant, preferably from the medium and/or supernatant.

As outlined above, albeit the amino acid sequence of the purified polypeptides may be the same, the exact glycosylation pattern may vary between individual polypeptides, due to the nature of the glycosylation process in a bacteria. Thus, in an embodiment, the polypeptide of interest and/or plurality of glycosylated polypeptides of interest is a polypeptide or pool of polypeptides according to the present invention.

In an embodiment, in step b), the glycosylated polypeptide is purified from the medium and/or supernatant, preferably in the absence of a lysis step. As shown in example 5, it is possible to purify the protein from the supernatant/medium.

### Product by Process

A further aspect of the invention relates to a glycosylated polypeptide and/or plurality of glycosylated polypeptides obtained by or obtainable by a process according to the invention. As also outlined in the example section, it can be difficult to precisely establish the glycosylation pattern of the individual polypeptides.

In an embodiment, the glycosylated polypeptide is a polypeptide according to the invention or a composition comprising the plurality of polypeptides with a glycosylation pattern according to the invention.

### Medical Uses

As outlined through-out the application, the polypeptides or compositions according to the invention are efficient vaccines (see Example 4—Pig challenge study). Thus, an aspect of the invention relates to the polypeptide and/or the composition according to the invention for use as a medicament, such as a vaccine.

In a similar aspect the invention relates to the polypeptide and/or the composition according to the invention is for raising an immune response in a patient, such as being a vaccine.

In yet an aspect the invention relates to the polypeptide and/or the composition according to the invention for use in the treatment, prevention and or alleviation of E. coli infections.

In an embodiment, the E. coli infection is an extra-intestinal pathogenic E. coli (ExPEC) infection.

In an embodiment, the polypeptide or the composition is for use in the treatment, prevention and or alleviation extraintestinal pathogenic E. coli (ExPEC) infection, such as an UPEC infection, such as urinary tract infections (UTIs), such as bladder infection and/or kidney infection. Again, as shown in Example 4—Pig challenge study, the polypeptide is highly efficient against bladder infections. Without being bound by theory it is believed that the glycosylation pattern of the polypeptide according the invention resembles the glycosylation pattern of the pathogenic strain to a higher degree by being produced in a non-pathogenic version of the WT strain. Thus, the polypeptide is not produced in a standard E. coli production strain.

In an embodiment, the polypeptide or the composition is for use in preventing an extraintestinal pathogenic E. coli (ExPEC) infection such as an UPEC infection from entering the bloodstream. In a related embodiment, the polypeptide and/or composition according to the invention is for use in the treatment, prevention and/or alleviation of sepsis.

In a preferred embodiment, the polypeptide or the composition is for use in the treatment, prevention and or alleviation of an UPEC infection.

### Antibody

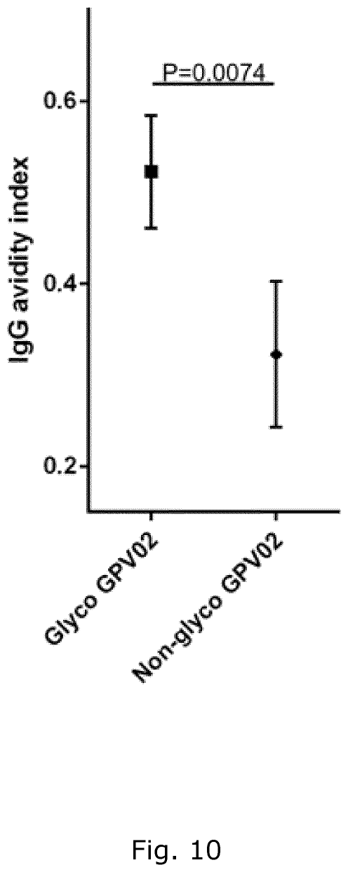

As outlined in Example 6—Antibody data and shown in FIG. 10, immunization with a hyperglycosylated polypeptide and/or composition according to the invention induces production of antibodies with higher avidity compared to a non-glycosylated versions isolated from a conventional E. coli expression strain e.g., BL21 (DE3) or MG1655.

Thus, in an aspect the invention relates to an antibody specific for the polypeptide according to the invention.

In an embodiment, the antibody is selected from the group consisting of a polyclonal antibody, a monoclonal antibody, an antibody wherein the heavy chain and the light chain are connected by a flexible linker, an Fv molecule, an antigen binding fragment, a Fab fragment, a Fab′ fragment, a F(ab′)₂ molecule, a single domain antibody (sdAB), such as a nanobody, a fully human antibody, a humanized antibody, and a chimeric antibody.

The antibodies may be suitable to generate chimeric and/or human versions that could be appropriate for human in vivo use.

Thus, the invention is also concerned with the polypeptides as described herein for use in animals to produce antisera such as for diagnostic and therapeutic purposes.

Antibodies obtained from animals exposed to the polypeptides as described herein, may be used for the treatment or diagnosis of a bacterial infection, such as an UPEC infection.

Thus, in yet an aspect the invention relates to in vitro uses of the polypeptides, compositions and antibodies according to the present invention.

In a related aspect the invention relates to the use of the glycosylated polypeptides according to the invention for raising antibodies against the glycosylated polypeptides.

### Other Aspects

An aspect of the invention relates to a method for immunizing a subject, the method comprising administering to the subject the polypeptide according to the invention or a composition according to the invention.

Another aspect relates to a method for treating a subject, which is infected with UPEC comprising administering to the subject the polypeptide and/or a composition according to the invention and/or an antibody according to the invention.

In an embodiment, said subject is a mammal, such as life stock, pets or racing animals, preferably a human.

It should be noted that embodiments and features described in the context of one of the aspects of the present invention also apply to the other aspects of the invention.

Throughout this specification, unless the context requires otherwise, the word “comprise” or variations such as “comprises” or “comprising”, will be understood to imply the inclusion of a stated integer or group of integers but not the exclusion of any other integer or group of integers.

### Items of the Invention

1. A polypeptide comprising:
   a) an amino acid sequence according to SEQ ID NO: 1; and/or
   b) an amino acid sequence having at least 80% sequence identity to SEQ ID NO: 1; and/or
   c) an amino acid sequence which is a fragment of at least 1000 consecutive amino acids from SEQ ID NO: 1; and/or
   d) an amino acid sequence having at least 80% sequence identity to SEQ ID NO: 1 and including a fragment of at least 1000 consecutive amino acids from SEQ ID NO: 1;
wherein said polypeptide has a glycosylation pattern defined by being glycosylated on at least 15 positions in SEQ ID NO: 1, selected from the group depicted in Table 2A-B, such as at least 20 positions or such as at least 40 positions.
2. The polypeptide according to item 1, being glycosylated on at least 15 positions such as on at least 20 positions, such as all of the positions depicted in Table 2B.
3. The polypeptide according to any of the preceding items, being glycosylated on at least 15 positions selected from the group depicted in Table 3A-B, such as on at least 20 positions, such as on at least 30 positions, or such as all of the positions according to Table 3A-B;
and/or
being glycosylated on at least 10 positions selected from the group depicted in Table 4, such as on at least 15 positions, such as on at least 20 positions or such as all of the positions depicted in Table 4.
4. The polypeptide according to any of the preceding items, being glycosylated on at least position S152, and/or S154 and/or S164, such as S152 and S154, such as S154 and S164, such as S152 and S164 or such as S152, S154 and S164;
and
being glycosylated on at least positions T592, and/or S594 and/or S597, such as T592 and S594, such as S594 and S597, such as T592 and S597, or such as T592 and S594 and S597.
5. A polypeptide comprising:
   a) an amino acid sequence according to SEQ ID NO: 1; wherein said polypeptide has a glycosylation pattern defined by being glycosylated on at least 10 positions selected from the group depicted in Table 4, such as on at least 15 positions, such as on at least 20 positions or such as all of the positions depicted in Table 4.
6. A composition comprising the polypeptide according to any of items 1-5 and/or a composition comprising a plurality of polypeptides with a glycosylation pattern according to any items 1-5.
7. A genetically modified E. coli which does not express:
   FimH;
   PapG; and
   WaaL.
8. The genetically modified E. coli according to item 7 being an ExPEC, such as UPEC, such as UTI89.
9. The genetically modified E. coli according to any of items 7-8, being avirulent and/or non-pathogenic and/or not capable of causing disease.
10. A process for producing a glycosylated polypeptide of interest and/or plurality of glycosylated polypeptides of interest, the process comprising
   a) expressing the polypeptide of interest in the genetically modified E. coli according to any of items 7-9; and
   b) purifying the glycosylated polypeptide of interest and/or plurality of glycosylated polypeptides of interest from said bacteria, such as from the lysate and/or medium and/or supernatant, preferably from the medium and/or supernatant.
11. A glycosylated polypeptide and/or plurality of glycosylated polypeptides obtained by or obtainable by a process according to item 10.
12. The polypeptide according to any of items 1-5 and/or the composition according to item 6 and/or the glycosylated polypeptide and/or plurality of glycosylated polypeptides according to item 11 for use as a medicament, such as a vaccine.
13. The polypeptide according to any of item 1-5 or the composition according to item 6 and/or plurality of glycosylated polypeptides according to item 11 for use in the treatment, prevention and/or alleviation of an E. coli infection, such as an extra-intestinal pathogenic E. coli (ExPEC) infection, such as an UPEC infection.
14. The polypeptide according to any of item 1-5 or the composition according to item 6 and/or plurality of glycosylated polypeptides according to item 11 for use in the treatment, prevention and/or alleviation of an UPEC bladder infection.
15. An antibody specific for a polypeptide according to any of items 1-5.

All patent and non-patent references cited in the present application, are hereby incorporated by reference in their entirety.

The invention will now be described in further details in the following non-limiting examples.

### EXAMPLES

### Example 1—Materials and Methods

### Bacterial Strains and Growth Conditions

Escherichia coli UTI89 (Infect Immun. 2001, 69:4572) was used as wildtype UPEC strain and is the basis of the production strain. For cloning purposes, strains were routinely grown aerated at 37° C. in Luria Bertani (LB) medium supplemented with 100 μg/ml ampicillin (Amp), 40 μg/ml kanamycin (Kan) or 30 μg/ml chlorampenicol (Cml) when necessary.

For production of full length YGHJ, all strains were grown in LB+M9 medium (Clark and Maaløe, J Mol Biol. 1967, 23:99) supplemented with 0.2% glucose and 0.4% casamino acids (1:1 vol/vol).

For the adhesion assay, we used UTI89 yghJ::3×FLAG (Kan^{R}) as the wildtype and compared to UT189 ΔwaaL ΔfimH ΔpapG ygh_::3×FLAG-10×His:: FRT (the production strain as defined in this invention) and E. coli K-12 MG1655 (non-pathogenic conventional production strain) carrying a pNDM220 (Mol Microbiol. 1998, 29:1065) derivative or pKD46, respectively. Strains were grown statically overnight at 37° C. in LB medium prior to the adhesion assay.

For pig challenge studies, UTI89 wildtype was grown overnight, statically in LB medium at 37° C. The culture was diluted 4000× in fresh LB medium and incubated overnight, statically at 37° C. Serial passage and static growth ensures that most of the population expresses type 1-pili necessary for establishing an infection (Hung et al. Nat Protoc. 2009, 4:1230).

### DNA Manipulations

### His-Tagging

The UTI89 yghJ gene was tagged at the 3′ end by the method described by Uzzau et al. (Proc Natl Acad Sci USA 2001, 98:15264). Briefly, a linear dsDNA molecule is prepared by PCR, which carries the epitope tag followed by a kanamycin resistance cassette (kan) flanked by FRT sites (FLP recombinase recognition sites; allow removal of drug resistance once the tagged clone is obtained). The tag-FRT-kan-FRT product is flanked by sequences (60-400 bp) that are homologues to the last part of yghJ and the region immediately downstream from the gene. The linear PCR product is transformed into the target strain, which carries an easily curable helper plasmid (pKD46) expressing the phage λ Red recombinase, that facilitates recombination between the homologues regions. Clones expressing the C-terminal-tagged YGHJ protein can be identified by standard immuno-detection methods (e.g. Western blotting), and the antibiotic resistance removed by means of a curable helper plasmid, pCP20, expressing the FLP recombinase (Proc Natl Acad Sci USA 2000, 97:6640). Primer sequences used to generate the yghJ-6×His, −3×FLAG-6×His and −3×FLAG-10×His clones are shown in Table 1.

### Deletions

The Δc4349-4351 mutant was generated by the Datsenko and Wanner method (Datsenko and Wanner, 2000) which, similar to epitope-tagging described above, utilizes phage λ Red recombinase and a linear PCR product carrying an antibiotic resistance gene (cml) flanked by sequences homologous to chromosomal regions up- and downstream of the area to be deleted. The recombination event, thus, replaces the deleted area on the chromosome by the cml cassette. Additionally, we used this technique in an attempt to delete the O-antigen cluster either fully (c2303-c2312) or partially (c2308-c2312 or c2303-c2307), however, these efforts were unsuccessful. Primer sequences are shown in Table 1.

The ΔwaaL, ΔfimH and ΔpapG mutations were created using a CRISPR/Cas9 procedure adapted from Zhao et al. (Microb Cell Fact. 2016, 15:205). Briefly, this method relies on a single plasmid encoding the Cas9 endonuclease and sgRNA necessary for generating a double stranded break in the target sequence; furthermore, the plasmid carries the donor DNA (homologous sequences flanking the target region), phage λ Red recombinase and recA genes required for DNA repair by homologous recombination. Because of a modular plasmid design, it is only necessary to prepare a custom guide sequence (N20, made by annealing two DNA oligos) and donor DNA (approx. 300 bp homology arms, made by PCR) for each target. The invariable components of the plasmid are amplified by PCR (part 1 and part 2) and the four parts are assembled by Golden Gate Assembly with BsaI restriction enzyme and T4 DNA ligase. Primers and oligos used to construct the deletion mutants are shown in Table 1.

### Cell Line Culture Conditions

The human bladder cell line ATCC 5637 was used to study the adherence capacity of the production strain. Cells were maintained in a humidified atmosphere containing 5% CO₂ at 37° C. and grown in Gibco RPMI 1640 medium (ATCC modification) supplemented with 10% heat-inactivated fetal bovine serum (FBS; Gibco) and Penicillin-Streptomycin (Pen-Strep; 100 units/ml; 100 μg/ml) (Gibco). When reaching 90% confluence the 5637 cells were trypsinized for 5 min, diluted 1:4 and seeded in 12-well plates (Nunc).

### Adhesion Assay

One hour prior to addition of bacteria, 100% confluent 5637 cells (approx. 5×10⁵ cells per well) were washed thrice with 1× phosphate buffered saline (PBS) and incubated with growth medium without Pen-Strep. A multiplicity of infection (MOI) of 0.5 was used. Bacteria and cells were incubated for 1 h. Non-adherent bacteria were removed by washing the cells thrice with PBS, vigorously shaking the plate on a plate-shaker on each washing step. Relative adhesion potential was determined by serial dilutions and plating onto selective LA plates. CFU was determined next day, and the adhesion ability of each strain was normalized to the adhesion ability of wild-type strain UTI89. The assay was done in four independent biological replicates, with four wells per strain. Each well was serial diluted, and dilutions spotted twice on selective plates.

### Western Blotting

Denaturing SDS PAGE and western blotting was carried out as described by Thorsing et al.(Thorsing et al., 2021). Antibodies used are specified in figure descriptions.

### Protein Purification

Glycosylated YGHJ was purified by His-purification essentially as described by Riaz et al. (Riaz et al., 2021) with minor changes. Filtered culture supernatant was adjusted to 200 mM NaCl and 0.05% Triton X-100 and incubated with TALON® resin to capture tagged YGHJ. After incubation, the resin was washed twice with wash buffer I (1× PBS, pH 7.2, containing 600 mM NaCl and 0.05% Triton X-100) and once with wash buffer II (0.5× PBS, containing 0.05% Triton X-100, [pH 7.6]). Glycosylated YGHJ was eluted by incubating the resin in elution buffer (0.5× PBS, containing 150 mM imidazole and 0.05% Triton X-100, [pH 9.4]). Eluates were spin filter concentrated and purified further by ion exchange on a Toyopearl NH2-750F 1 ml column (Tosoh Bioscience) using an AKTA start (Cytiva) chromatography system. The concentrated eluate was loop loaded onto the column, washed with 10 column volumes (CV) of buffer A (20 mM sodium phosphate buffer, pH 6.0, 75 mM NaCl, 0.01% Triton X-100), then 10 CV of buffer A with additional 260 mM NaCl, and eluted with 14 CV buffer A with additional 1.425 M NaCl. Purified proteins were quantified using the BCA Micro Assay (Thermo Fischer Scientific) relative to a bovine serum albumin standard.

TABLE 1
DNA primers and oligonucleotides used for construction of the
production strain.

| SEQ ID | | |
|---|---|---|
| NO: | Primer sequence | Application |
| 2 | AAACCGGAAAAAGGGCCGGAAACCATTAACAA | yghJ-6xHis tagging |
| | GGTTACCGAGCATAAGATGTCTGCCGAGCACC | |
| | ACCATCATCACCATTAGTAA | |
| 3 | TTGACCCGATGCGCCTTATATCATGCCGGATGC | yghJ-6xHis tagging |
| | GGCGTGAACGCCTTATCCGGCCTACAGGCCAT | |
| | ATGAATATCCTCCTTAGTTCC | |
| 4 | CAAACAGTGGTATCCAGATGGTG | yghJ-3xFLAG-His |
| | | tagging, fragment A |
| 5 | TTATCGTCGTCATCTTTGTAGTCG | yghJ-3xFLAG-His |
| | | tagging, fragment A |
| 6 | GACTACAAAGACCATGACGGTGATTATAAAGAT | yghJ-3xFLAG-6xHis |
| | CATGATATCGACTACAAAGATGACGACGATAAA | tagging, fragment B + |
| | CACCACCATCATCACCATTAGTAA | primer GPV187 |
| 7 | GACTACAAAGACCATGACGGTGATTATAAAGAT | yghJ-3xFLAG-10xHis |
| | CATGATATCGACTACAAAGATGACGACGATAAA | tagging, fragment B + |
| | CACCACCATCATCACCACCATCATCACCATTAG | primer GPV187 |
| | TAATGTAGG | |
| 8 | TAGGCCGGATAAGGCGTTC | yghJ-3xFLAG-His |
| | | tagging, fragment C |
| 9 | GCTTATTTTTGACTGCGTACTCG | yghJ-3xFLAG-His |
| | | tagging, fragment C |
| 10 | TTGTTCTACAACCTGTCGCCCGTCAATCAGCGT | c4349-4351 deletion |
| | ACGGTAATTGCGACCTTGTTGAACTACTTCTCC | |
| | TGATGTGTAGGCTGGAGCTGCTTCG | |
| 11 | GCATATCACGCCAACCAATCAACTGTAAGCCAC | c4349-4351 deletion |
| | GCAGCGTATAGGTCGGTGCCGTGGTGTTGTTA | |
| | TTCATCATATGAATATCCTCCTTAGTTCC | |
| 12 | TACCTCTATTAATCAAACTGAGAGCCGCTTATTT | C2308-2312 deletion |
| | CACAGCATGCTCTGAAGTAATATGGAATAAATT | |
| | AAGGTGTAGGCTGGAGCTGCTTCG | |
| 13 | AATATATATATCATTATAAATTGTCACCTACCCA | C2308-2312 deletion |
| | AATAAAGCCATGAGTTATTAACTTGCATTTTGAA | |
| | TTCATATGAATATCCTCCTTAGTTCC | |
| 14 | ATATTAATAAATAAAATTCAAAATGCAAGTTAAT | C2303-2307 deletion |
| | AACTCATGGCTTTATTTGGGTAGGTGACAATTT | |
| | ATAGTGTAGGCTGGAGCTGCTTCG | |
| 15 | TTGCAATAAAAAAACTGTAATTTTATTTTCACTT | C2303-2307 deletion |
| | TGAAAAACCTGTTCTTTTTTAACTTTTCGGTTTC | |
| | ATCATATGAATATCCTCCTTAGTTCC | |
| 16 | CCAGGTCTCAGCTCTGCTGAATGGAAGCTTGG | CRISPR/Cas “part 1” |
| | ATTCTCACC | PCR |
| 17 | CCAGGTCTCACGCTTAAGATCTGACTCCATAAC | CRISPR/Cas “part 1” |
| | AGAGTACTCGCC | PCR |
| 18 | CCAGGTCTCAGTTTTAGAGCTAGAAATAGCAAG | CRISPR/Cas “part 2” |
| | TTAAAATAAGGC | PCR |
| 19 | CCAGGTCTCAGCACCACAGGCCCATGGATTCTT | CRISPR/Cas “part 2” |
| | CG | PCR |
| 20 | AGCGCGTCTGGCAATGTATGAAGT | waal N20 guide |
| | | sequence |
| 21 | AAACACTTCATACATTGCCAGACG | waal N20 guide |
| | | sequence |
| 22 | CCAGGTCTCAGTGCTGGAATGTTCTACTTAATG | Awaal donor DNA, “up” |
| | GCGA | fragment |
| 23 | AGTCGGAACCGGTTTAGAAAAGAGTGCAACAT | Awaal donor DNA, “up” |
| | CATCCCACAAAGAC | fragment |
| 24 | GTCTTTGTGGGATGATGTTGCACTCTTTTCTAA | Awaal donor DNA, |
| | ACCGGTTCCGACT | “down” fragment |
| 25 | CCAGGTCTCAGAGCATAGACCCAATATCACGGT | Awaal donor DNA, |
| | TTATGG | “down” fragment |
| 26 | AGCGAGGGTAGTCCGGCAGAGTAA | fimH N20 guide |
| | | sequence |
| 27 | AAACTTACTCTGCCGGACTACCCT | fimH N20 guide |
| | | sequence |
| 28 | CCAGGTCTCAGTGCCCGCGCAAAACATCCAGT | AfimH donor DNA, “up” |
| | | fragment |
| 29 | ACAAAAGTCACGCCAATAATCGAATTTACCGAC | AfimH donor DNA, “up” |
| | CAGCCCATCA | fragment |
| 30 | TGATGGGCTGGTCGGTAAATTCGATTATTGGCG | AfimH donor DNA, |
| | TGACTTTTGT | “down” fragment |
| 31 | CCAGGTCTCAGAGCTCCGTGGTTGGGCTGATT | AfimH donor DNA, |
| | AT | “down” fragment |
| 32 | AGCGGATAATTACTATCTTACCAA | papG N20 guide |
| | | sequence |
| 33 | AAACTTGGTAAGATAGTAATTATC | papG N20 guide |
| | | sequence |
| 34 | CCAGGTCTCAGTGCACTCATTTTGGTATAGCGC | ApapG donor DNA, “up” |
| | TGT | fragment |
| 35 | GAAAGAGTCTGAGCCGCATAAGCATCATTACAG | ApapG donor DNA, “up” |
| | CCTGACAG | fragment |
| 36 | CTGTCAGGCTGTAATGATGCTTATGCGGCTCAG | ApapG donor DNA, |
| | ACTCTTTC | “down” fragment |
| 37 | CCAGGTCTCAGAGCCACAACACACTATCCGGCT | ApapG donor DNA, |
| | C | “down” fragment |

### Example 2—Production Dependent Glycosylation Pattern

### Aim of Study

To demonstrate that purification of full length YGHJ from the detoxified and avirulent production strain results in efficient protein glycosylation (GPV02, as defined in this invention). Further to demonstrate that, in contrast, if full length YGHJ is isolated from a conventional E. coli production strain e.g. BL21 (DE3) or MG1655 this results in a protein with significantly fewer glycosylations and a different glycosylation pattern compared to the production strain according to the present invention.

### Materials and Methods

Full length glycosylated YGHJ (GPV02) was isolated from the production strain according to the invention and compared to full length YGHJ, which was isolated from a conventional E. coli over-expression strain (Conventional YGHJ antigen). The BEMAP protocol was used to identify O-linked glycosylated tryptic peptides within the two YGHJ protein variants (Boysen et al., 2016). For the BEMAP analysis of GPV02, 3 μg to 138 μg purified protein was used as sample input. For the BEMAP analysis of the conventional YGHJ antigen, 300 μg purified protein was used as sample input. The relative abundance of each of the identified glycopeptides within a BEMAP analysis was also determined using the Minora Feature Detector of the Proteome Discoverer software program. Standard settings of the Minora tool were used. Briefly described, the Minora tool measures the area under each identified glycopeptide peak. By summing all the areas, the relative abundance for each peptide can be calculated as a percentage (area of a peptide/total areas of all peaks*100).

### Results

A total of 12 BEMAP analyses using different sample amounts of GPV02 were performed to identify glycopeptides. For GPV02, we observed that the number of identified glycopeptides correlated with an increasing amount of sample used in the BEMAP analysis. For example, where 9.6 μg, 31.8 μg, 82 μg and 138 μg GPV02 sample was used as input 16, 39, 54 and 66 glycopeptides were identified, respectively. For a subset of glyco-peptides, the same glycosylation pattern was observed irrespectively of sample amount used for the BEMAP analysis. For another subset of glycopeptides, these were only identified when the highest amounts of sample were used for the analysis. By combining all the BEMAP analyses of GPV02, a total of 102 modified residues were identified, see table 2AB and further glycosylation patterns were established, see table 3AB and Table 4.

TABLE 2
Glycosylation sites identified in GPV02 (SEQ ID NO: 1)
Group 1 (102 sites)
A) Glycosylation sites also found in ETEC (61 (sites)

| | | | | | | |
|---|---|---|---|---|---|---|
| S99 | T103 | T106 | S111 | S112 | T116 | T118 |
| T122 | S180 | S177 | T229 | S356 | T357 | T358 |
| T363 | T422 | S438 | T461 | S467 | S475 | T476 |
| S481 | T482 | S494 | S552 | S580 | T592 | S594 |
| S597 | T592 | S594 | S597 | S612 | S645 | S722 |
| S731 | S737 | S739 | S751 | S757 | S823 | S849 |
| T919 | S943 | T961 | S1003 | S1015 | S1016 | S1024 |
| S1086 | S1102 | T1105 | T1146 | S1161 | S1166 | S1191 |
| S1215 | T1230 | S1233 | S1234 | S1357 | | |

B) Glycosylation sites specific to UPEC (41 sites)

| | | | | | | |
|---|---|---|---|---|---|---|
| S152 | S154 | S164 | S124 | T176 | S178 | S216 |
| T333 | T428 | S465 | S496 | T528 | S532 | T540 |
| T545 | S571 | S13 | T620 | T625 | T706 | S740 |
| T871 | T885 | T899 | S954 | T979 | S1060 | S1065 |
| S1971 | S1074 | T1087 | S1104 | T1218 | S1427 | T1428 |
| S1438 | T1445 | S1451 | S1470 | S1488 | T1508 | |

TABLE 3
Selected glycosylation sites identified in YGHJ (SEQ ID NO: 1)
Group 2 (41 sites)
A) Glycosylation sites from UPEC
signature peptides (15 sites)

| | | | | | | |
|---|---|---|---|---|---|---|
| T103 | T106 | S111 | S112 | T116 | T118 | T122 |
| S177 | S494 | T592 | S594 | S597 | S1102 | T1105 |
| S1215 | | | | | | |

B) Glycosylation sites specific to
UPEC and UPEC signature peptides (26 site)

| | | | | | | |
|---|---|---|---|---|---|---|
| S124 | S152 | S154 | S164 | T176 | S178 | S216 |
| T428 | S496 | T528 | S532 | T540 | T899 | T979 |
| S1065 | S1071 | S1074 | S1104 | T1218 | S1427 | T1428 |
| S1438 | T1445 | S1451 | S1470 | S1488 | | |

TABLE 4
Selected glycosylation sites identified in YGHJ (SEQ ID NO: 1)
Group 3 (25 sites)
Glycosylation sites specific to UPEC and UPEC-only signature peptides

| | | | | | | |
|---|---|---|---|---|---|---|
| S152 | S154 | S164 | S216 | T428 | T528 | S532 |
| T540 | T899 | T979 | S1065 | S1071 | S1074 | S1427 |
| T1428 | S1438 | T1445 | S1451 | S1470 | S1488 | S496 |
| T1218 | S1104 | T176 | S178 | | | |

In order to compare the glycosylation pattern of GPV02 with Conventional YGHJ antigen (such as produced and tested in Nesta et al. (Nesta et al., 2014)), one BEMAP analysis of Conventional YGHJ antigen was made.

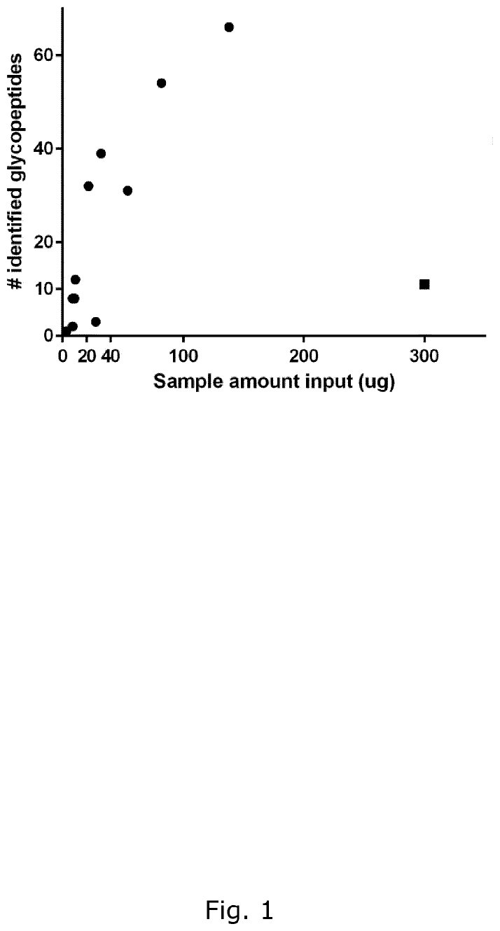

To maximize the number of identified glycopeptides originating from the Conventional YGHJ antigen, 300 μg protein was used as input to the BEMAP protocol. However, despite this high amount of protein, the BEMAP analysis resulted in the identification of only 11 glycopeptides. The correlation between the number of identified glycopeptides (for both GPV02 and conventional YGHJ antigen) and sample amount was plotted and shown in FIG. 1.

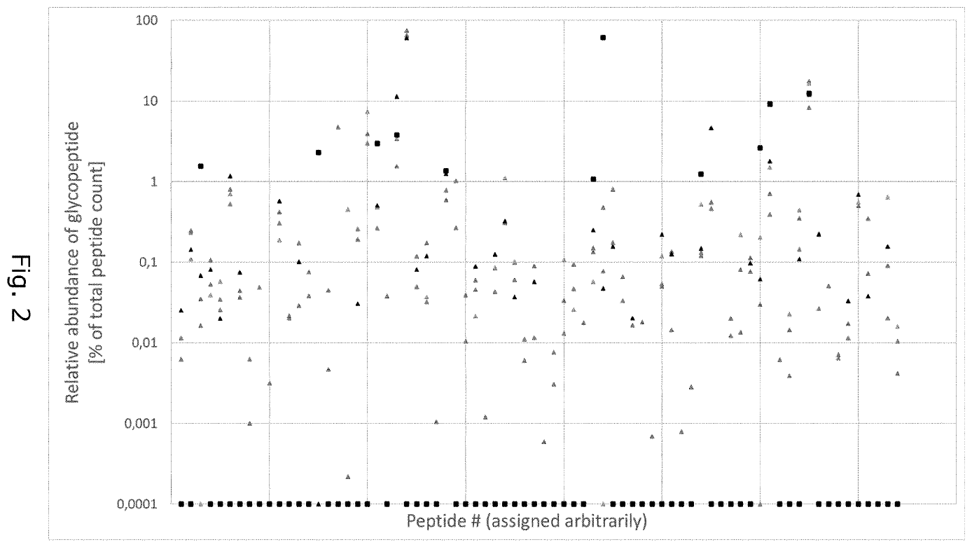

Using Minora Feature Detector software tools, we determined the relative abundance of all identified glycopeptides derived from GPV02. As mentioned above, a small subset of peptides was almost always identified when analysing GPV02. These sequences were also the ones with the highest abundance. For example, and as shown in FIG. 2, in four BEMAP analyses where 31.8 μg, 54 μg, 82 μg and 138 μg GPV02 sample was used as input 39, 31, 54 and 66 glycopeptides were identified, respectively. In these four analyses, shown in FIG. 2, the relative abundance of two peptides was on average 66% and 13%, respectively. In the analysis, it was also determined that five peptides had an abundance above 1%, whereas the remaining peptides were below. Some peptides were found to have a relative abundance close to 0.001%. We highlight that the dynamic range of the analysis spans five orders of magnitude. For comparison we also determined the relative abundance of the 11 identified glycopeptides derived from the conventional full length YGHJ, see FIG. 2. The relative abundance of these peptides was different from what had been determined when analysing GPV02. For example, the peptide with an average abundance of 66% in GPV02, was not detected at all in the sample derived from the conventional full length YGHJ. Conversely, one peptide derived from the conventional full length YGHJ that had an abundance of 61.4%, was determined to be approximately 400-fold less abundant in GPV02. At an overall level, the analysis shows that the abundance of eight peptides out of 11 sequences were dissimilar when comparing the two antigens and only three peptides were similar.

### Conclusion

GPV02 is a 1520 amino acid polypeptide which contains 237 Ser/Thr residues. Using 138 μg GPV02 a total of 102 modified residues were identified (Table 2). This number shows that GPV02 is hyper-glycosylated. It is unlikely that all 237 residues are modified, as many Ser/Thr sites are buried within the tertiary structure and therefor inaccessible for the glycosyltransferases.

This is supported by the BEMAP analysis depicted in FIG. 2 which indicates that there seems to be an upper limit for the number of sites that can be modified. The result of the glycopeptide analysis of the conventional full length YGHJ antigen as produced in Nesta et al. (Nesta et al., 2014) was very surprising. Only 11 peptides were identified even though 300 μg protein sample was used as input. One would have expected to identify at least a similar number of glycopeptides when comparing to GPV02 in the analysis where the highest amount of sample had been used. This was however not the case and indicates that the choice of strain used for protein antigen production is important when specifically looking for protein glycosylation. The relative glycopeptide abundance was determined for the two antigens. Surprisingly, the relative abundance and pattern was very dissimilar indicating that the protein glycosylation machinery must be functioning mechanistically different in the two strains.

It is concluded that GPV02 protein glycosylation heavily depends on the choice of host strain. This has serious implications from a production point of view. It is generally not considered desirable to express immunogens in the natural pathogenic host due to the risk of infections for the personal handling the purification process, waste management and post-production instrument handling. Therefore, to obtain a glycosylated GPV02 vaccine, as defined in this invention, one must engineer a production strain, which is able to produce the antigen with the glycosylation patterns according to the natural pathogenic bacteria and at the same time is non-pathogenic allowing it to be grown in low level biosafety laboratories. We have shown that this can be achieved by deleting genes involved in Lipopolysaccharide production as well as colonization of human cells (Example 3).

### Example 3—Non-Pathogenic Production Strain

### Aim of Study

To obtain a non-pathogenic UPEC strain for production of full length YGHJ antigen (tagged to enable purification), which retains its ability to effectively glycosylate YGHJ. Genes for editing were chosen based on their importance in pathogenicity as well undesired immunogenicity. We focused on the elimination of adhesins to hamper the pathogen's ability to establish adhesion to host cells as well as reduction in the pyrogenic O-antigen of the pathogen.

### Materials and Methods

His-Tagging:

The UTI89 yghJ gene was tagged at the 3′ end by the method described by Uzzau et al. 2001. Briefly, a linear dsDNA molecule is prepared by PCR, which carries the epitope tag followed by a kanamycin resistance cassette (kan) flanked by FRT sites (FLP recombinase recognition sites; allow removal of drug resistance once the tagged clone is obtained). The tag-FRT-kan-FRT product is flanked by sequences (60-400 bp) that are homologues to the last part of yghJ and the region immediately downstream from the gene. The linear PCR product is transformed into the target strain, which carries an easily curable helper plasmid (pKD46) expressing the phage λ Red recombinase, that facilitates recombination between the homologues regions. Clones expressing the C-terminal-tagged YGHJ protein can be identified by standard immuno-detection methods (e.g. Western blotting), and the antibiotic resistance removed by means of a curable helper plasmid, pCP20, expressing the FLP recombinase (Datsenko and Wanner, 2000). Primer sequences used to generate the yghJ-6×His, −3×FLAG-6×His and −3×FLAG-10×His clones are shown in Table 1.

Deletions:

The Δc4349-4351 mutant was generated by the Datsenko and Wanner method (Datsenko and Wanner, 2000), which, similar to epitope-tagging described above, utilizes phage λ Red recombinase and a linear PCR product carrying an antibiotic resistance gene (cat) flanked by sequences homologous to chromosomal regions up- and downstream of the area to be deleted. The recombination event, thus, replaces the deleted area on the chromosome by the cat cassette. Additionally, we used this technique in an attempt to delete the O-antigen cluster either fully (c2303-c2312) or partially (c2308-c2312 or c2303-c2307), however, these efforts were unsuccessful. Primer sequences are shown in Table 1.

The ΔwaaL, ΔfimH and ΔpapG mutations were created using a CRISPR/Cas9 procedure adapted from Zhao et al. (Zhao et al., 2016). Briefly, this method relies on a single plasmid encoding the Cas9 endonuclease and sgRNA necessary for generating a double stranded break in the target sequence; furthermore, the plasmid carries the donor DNA (homologous sequences flanking the target region), phage λ Red recombinase and recA genes required for DNA repair by homologous recombination. Because of a modular plasmid design, it is only necessary to prepare a custom guide sequence (N20, made by annealing two DNA oligos) and donor DNA (approx. 300 bp homology arms, made by PCR) for each target. The invariable components of the plasmid are amplified by PCR (part 1 and part 2) and the four parts are assembled by Golden Gate Assembly with BsaI restriction enzyme and T4 DNA ligase. Primers and oligos used to construct the deletion mutants are shown in Table 1.

### Results

For purification purposes, the yghJ gene of UTI89 was modified to encode YGHJ with a C-terminal 3×FLAG-10×His tag (see Example 5 below for choice of epitope tag). We achieved this by the Datsenko-Wanner method (Datsenko and Wanner, 2000), which leaves some “scar” sequence downstream of the edited region after FLP-mediated excision of the antibiotic selection marker, but other methods (e.g. CRISPR/Cas) could also be used to introduce the tag.

### O-Antigen Elimination

Deletion of wzx and wxy genes:

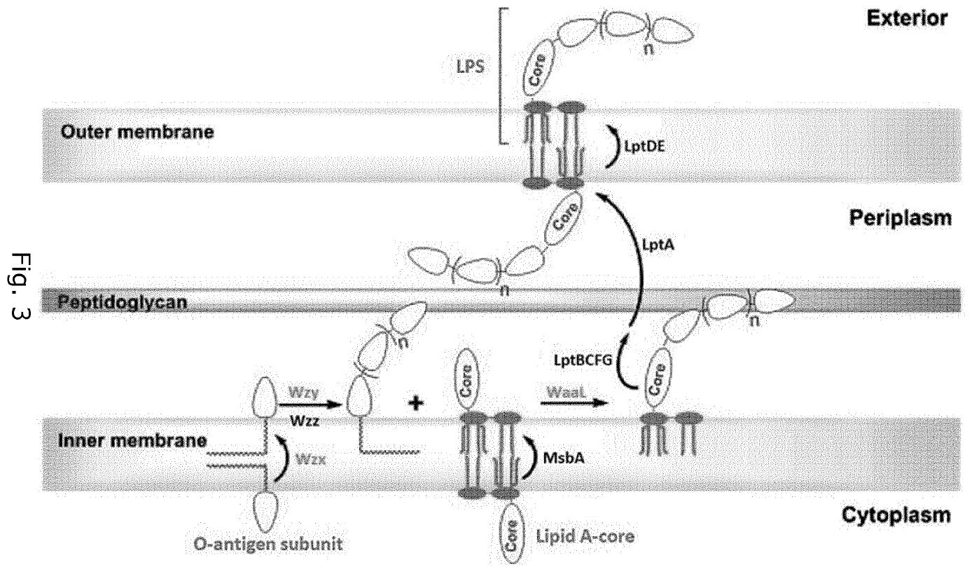

To eliminate O-antigen from the cell surface, we sought to delete the wzx and wxy genes involved in O-antigen transport and polymerization, respectively (FIG. 3). In E. coli, genes involved in O-antigen biosynthesis are generally found in a cluster between the ga/F and gnd genes (Samuel & Reeves, 2003), which is also the case for UTI89 (genes c2303-c2312, of which rfbE/c2308 is a putative 0-antigen transporter and c2307 encodes a putative O-antigen polymerase). However, our efforts to delete the entire cluster or part of it (c2308-c2312 or c2303-c2307) were unsuccessful, most likely as the deletion was lethal. Therefore, a new strategy was attempted: At a distant locus on the chromosome, wzxE (c4349) and wzyE (c4351) are annotated as a putative O-antigen flippase and putative O-antigen polymerase, respectively. We were able to delete both these genes along with c4350, encoding WecF involved in enterobacterial common antigen (ECA) biosynthesis. However, as shown in FIG. 3, the Δc4349-4351 mutant only displayed about 2-fold less O-antigen in the culture supernatant relative to the wildtype, which was inadequate for the further use of the mutant as a production strain.

WaaL deletion:

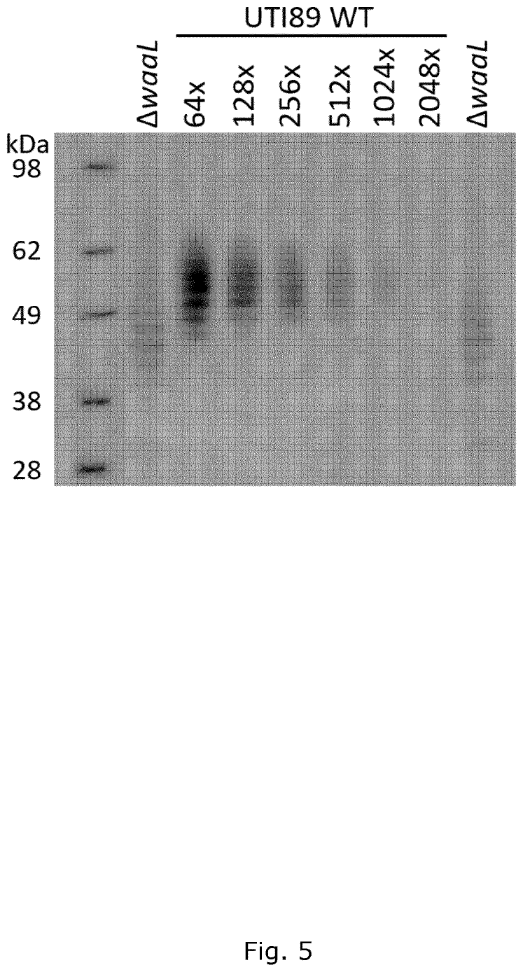

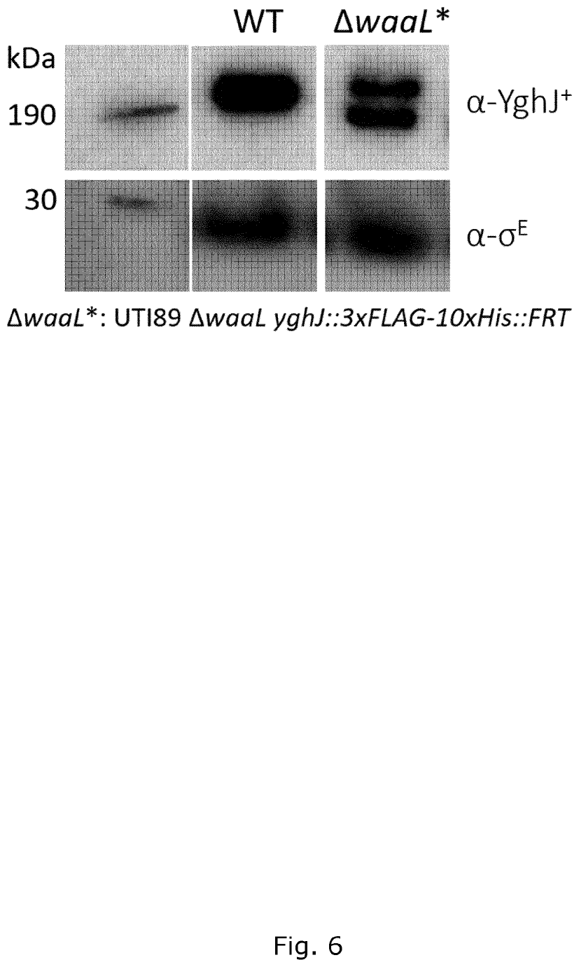

In an alternative approach, we made a deletion in the waaL gene (c4167), annotated as an O-antigen ligase (FIG. 3), using the CRISPR/Cas9 method that resulted in in-frame deletion of amino acids 35-385 of WaaL. This mutant has approx. 500-fold less O-antigen in the culture supernatant relative to the wildtype strain (FIG. 5), and a change in the migration pattern on the SDS gel indicates, that the remaining O-antigen polymers in the ΔwaaL mutant are generally shorter. Western blot analysis of culture supernatants using antibodies raised against glycosylated YGHJ showed, that the amount of exported YGHJ is comparable for wildtype and ΔwaaL mutant, but noticeably, YGHJ exported from the deletion mutant appears as two bands rather than one (FIG. 6, upper panel). Concerned that knock out of WaaL leads to outer membrane instability either directly, due to lack of 0-antigen, or indirectly, by hampering other biosynthetic pathways (e.g peptidoglycan, capsule, or ECA synthesis) that rely on recycling of the common undecaprenyl phosphate carrier (Figure. 3), we analysed the intracellular level of a^{E}, as an indicator of envelope stress, and found comparable levels in wildtype and ΔwaaL mutant (FIG. 6, lower panel).

Adhesins elimination:

Deletion of fimH (c5017) and papG (c4887):

The UT189 ΔwaaL ygh_::3×FLAG-10×His::FRT strain was further edited to reduce its pathogenicity potential by eliminating two adhesins, encoded by fimH (c5017) and papG (c4887), both of which are involved in initial establishment of infection. Using the CRISPR/Cas9 (Zhao et al., 2016) method we sequentially introduced in-frame deletions to remove amino acids 19-290 of FimH and amino acids 20-238 of PapG. When testing the resulting production strain (UTI89 ΔwaaL ΔfimH ΔpapG ygh_::3×FLAG-10×His::FRT) in an in vitro adhesion assay with human bladder cell line 5637, we found that adherence of the mutant was significantly reduced compared to the wildtype, and was even lower than the adherence of a non-pathogenic laboratory strain, E. coli K-12 MG1655 (FIG. 7).

### Conclusion

A production strain for expression of YghJ-3×FLAG-10×His was established based on the natural pathogenic host, E. coli UTI89. The production strain was rendered incapable of colonization by in-frame deletion of the genes encoding adhesins, FimH and PapG. The production strain was furthermore modified to attach less 0-antigen to lipopolysaccharides (LPS), which is the most prominent constituent of the outer leaflet of the outer membrane. This was achieved by deletion of the waaL gene, encoding O-antigen ligase, giving rise to a 500-fold reduction in 0-antigen levels compared to the wildtype (FIG. 5).

The phenotypic importance of these deletions was confirmed in an in vitro adhesion assay, in which the adherence of the production strain to human bladder cells was comparable to a non-pathogenic control and significantly lower than the wildtype strain (FIG. 7). The phenotype of the production strain as defined in this invention was characterized by not only reduced adherence compared to wild type and conventional production strain, but also when compared to single KO mutants ΔwaaL and ΔfimH (data not shown).

### Example 4—Pig challenge study

### Aim of Study

To assess the extent of protection afforded by GPV02, produced in the production strain according to the invention (see example 3) against an UPEC bladder infection in pigs.

### Materials and Methods

The experimental pig model (immunization and bladder infection) uses nine-week-old female LYD pigs which are challenge with UPEC UTI89 in the bladder (Nielsen et al., 2019). All pigs are challenged with a UPEC UTI89 dose of 1×10⁴ CFU's in a total volume of 100 ml PBS buffer, grown as described above. This dose has experimentally been determined to be the minimum dose required for 100% of the animals to get a bladder infection (Stærk et al., 2022). The experiment consisted of 40 pigs which were divided into two separate studies. In the first and second leg of the study, the experiment was stopped eight- and four-days post-infection, respectively.

The animals were immunized three times two weeks apart subcutaneously in the neck. If a Day 28 pre-defined antibody titre threshold was reached for the vaccinated pigs, all animals were to be inoculated on day 42. In each of the two studies 10 pigs were immunized with the GPV02 antigen or mock immunized with a saline buffer. When immunized with GPV02, the dose consisted of 25 ug GPV02 as well as 0.5 ug dmLT and 8 mg Litevax CMS adjuvant in a total volume of 1.5 ml. Urine samples were collected before inoculation, 1-day post-infection as well on the last day of the experiment. The number of CFU's in the urine as well as the number of bacteria associated with the bladder tissue were determined by serial dilutions and plate counting. IgG and IgA antibody end-point titres were determined in blood and vaginal swap samples isolated throughout the challenge study using ELISA where the plates had been coated with GPV02. A schematic outline of the challenge study is shown in Table 5.

TABLE 5
Outline of challenge study showing timepoints where different
types of samples are isolated, and tasks are performed.

| Tasks and | | | | | | Final |
|---|---|---|---|---|---|---|
| samples | Day 0 | Day 14 | Day 28 | Day 42 | Day 43 | Day |
| Immunization | x | x | x | | | |
| Blood | x | x | x | x | | x |
| Vaginal swap | x | x | x | x | | |
| Stop/Go decision | | | x | | | |
| Bladder infection | | | | x | | |
| Urine collection | | | | x | x | x |
| Bladder collection | | | | | | x |

### Results

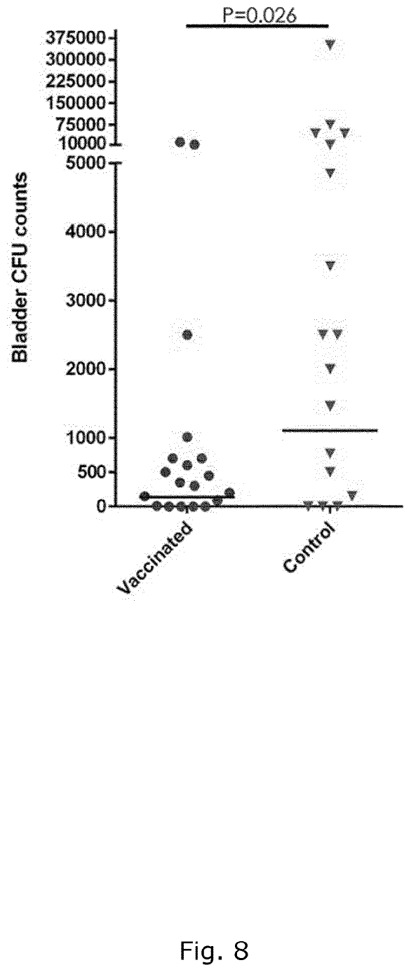

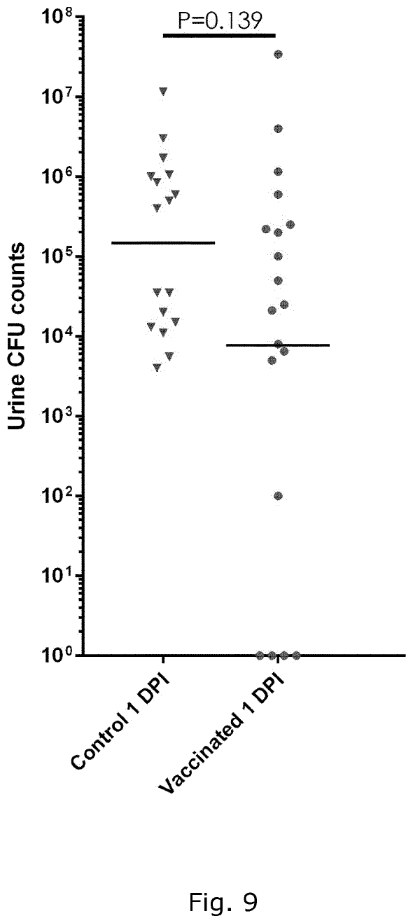

Immunization and bladder infection was carried out as described above. At Day 43 (1 day post infection (DPI)), the urine from three out 19 pigs in the vaccinated group did not contain bacteria demonstrating sterilizing immunity (P=0.168). In contrast, the urine samples of all the control group animals contained viable bacteria verifying a bladder infection. The number of bacteria associated with the bladder tissue was enumerated for both groups at the termination of the experiment. As seen in FIG. 8, GPV02 vaccination significantly reduces the ability of UTI89 to colonize the bladder tissue. The number of bacteria in the urine was determined 1 DPI for both groups. As shown in FIG. 9, the number of bacteria in the urine is reduced in the vaccinated animals compared to the control group.

### Conclusion

The presented data demonstrates the biological relevance of GPV02 as a vaccine candidate. GPV02 confers sterilizing immunity in 15.8% of the pigs against a bladder infection, significantly fewer CFU in bladder tissue upon termination as well as fewer urine CFU counts 1 DPI from the vaccinated group of animals compared to the control group, P=0.0026 and P=0.139, respectively.

In a previous study, the Full length YGHJ/SSLE antigen failed to protect against a bladder infection in a mouse model (Nesta et al., 2014). To the best of our knowledge the results of the present invention show for the first time a YGHJ/SSL derived antigen conferring protection in an animal model for urinary tract infection. The differentiating feature between GPV02 and the antigen used in Nesta et. al 2014 being hyper-glycosylation.

As outlined above, the antigen used in Nesta et al. is produced in a conventional production strain (and not its original host), which likely gives rise to suboptimal antigens, since the glycosylation pattern is different.

### Example 5—Tagging

For the purpose of antigen production from culture supernatants, the yghJ gene was edited to encode an in-frame polyhistidine tag (His-tag) at the end of the open reading frame, thus enabling purification on immobilized metal affinity chromatography resins, such as TALON® resin. As YGHJ carries an N-terminal signal peptide targeting the protein for export, the His tag was placed at the C-terminus.

Initially, we used a 6×His tag, but were not able detect any binding to TALON® resin under the conditions tested. Next, a triple FLAG tag was added between YGHJ and the 6×His tag, acting as a 22 amino acid spacer that provides some distance between YGHJ and the His-tag to make it more available for interaction with the resin. The YghJ-3×FLAG-6×His fusion bound to TALON® resin, although only weakly. Thus to increase the strength of the interaction, the production strain was edited further to encode ten histidine residues in the tag. This modification improved the interaction between YghJ-3×FLAG-10×His fusion and TALON® resin significantly (see Table 6). In a similar setup a construct using a 4×glycine-serine linker (GS linker) (four glycines followed by one serine), instead of a 3×FLAG tag, in front of the His-tag has been tested and shown to be efficient for purification purposes (Table 6). This setup has been tested since it is considered reducing linker immunogenicty”.

TABLE 6
Relative binding efficiency of C-terminal His tags to TALON ® resin
when purifying tagged YghJ (YGHJ) from complex culture supernatants.

| Protein | Purification from culture supernatant |
|---|---|
| YghJ-6xHis | − |
| YghJ-3xFLAG-6xHis | + |
| YghJ-3xFLAG-10xHis | +++ |
| YghJ-GS linker-10xHis | +++ |

### Conclusion

To improve the purification process an YghJ-3×FLAG-10×His fusion construct was prepared and used in the production stain as defined in this invention.

### Example 6—Antibody Data

### Aim of Study

To demonstrate that immunization with hyper-glycosylated GPV02 induces production of antibodies with higher avidity compared to non-glycosylated full length YGHJ version isolated from a conventional E. coli expression strain e.g., BL21 (DE3) or MG1655.

### Materials and Methods

Animal immunization:

A glycosylated GPV02 as well as a non-glycosylated full length YGHJ antigen was purified from their respective strains, using the protocol described (Thorsing et al., 2021). Nine-week-old female LYD pigs were used for the experiment. The study consisted of 8 pigs, which were divided into two groups of four animals each. The animals were immunized twice two weeks apart subcutaneously in the neck. The groups received GPV02 or non-glycosylated full length YGHJ variant. The dose consisted of 25 μg antigen as well as 0.5 μg dmLT and 8 mg Litevax CMS adjuvant in a total volume of 1.5 ml. Two weeks after the final dose, serum was isolated from the animals. Serum samples were used for IgG and IgA antibody end-point titres determination and the avidity assay.

Avidity assay:

The avidity assay is ELISA based and is carried out essentially as described in (Luo et al., 2016) with as few modifications. Briefly described, ELISA plates were coated with either 0.15 μg/ml GPV02 or 0.3 μg/ml non-glycosylated full length YGHJ in PBS buffer overnight at 4° C. Pre-immune sample start dilution was ×50 fold whereas the start serum sample dilution from the immunized animals was ×400 fold before being added to the plates. All sera samples were ×2 fold serially diluted in the plate. Serum samples were tested on plates coated with the same antigen as used for immunization, GPV02 and non-glycosylated full length YGHJ, respectively. After 1 hr of sera incubation at room temperature the plates were washed in PBS buffer (PBS+0.05% tween 20). PBS buffer with or without 6M urea was then added to the plates for 75 minutes at room temperature. The plates were washed in PBS buffer before ×16.000 fold diluted secondary Pig IgG-HRP conjugated antibody was added to the wells for 1 hr at room temperature. Finally, the plates were washed in PBS buffer before the responses were determined by kinetic ELISA. The avidity was calculated as Kinetic ELISA slopes (Vmax)+UREA/-UREA.

### Results

The avidity index was calculated for both groups of animals and plotted in FIG. 10. The analysis shows that pigs immunized with GPV02 produce antibodies with a significantly higher functional affinity towards the antigen compared to the animals vaccinated with the non-glycosylated full length YGHJ antigen.

### Conclusion

Memory B cells are a major component of the antibody-mediated long-term protective immunity following infection or vaccination. High antibody avidity has previously been shown to correlate with the presence of antigen-specific memory B cells in a number of human bacterial pathogens (Alam et al., 2013; Luo et al., 2016). Therefore, the antibody avidity could be a marker for predicting if a vaccine will provide protective immunity.

Our analysis shows that vaccination with a glycosylated antigen, such as GPV02, results in a significantly higher antibody avidity compared to a non-glycosylated, but otherwise identical variant. This result suggests that GPV02 isolated from the production strain could provide protective immunity to a higher extent than antigens currently produced in conventional production strains by the industry.

### Example 7—Glycan to Polypeptide Ratio

### Aim of Study

To further characterize YghJ isolated from a wild type UPEC strain, a standard E. coli K12 production strain and the production strain according to the invention, the services of the biotech company Spectralys Biotech has been used. Spectralys uses FTIR (fourier-transform infrared spectroscopy) to analyze proteins with respect to the glycan to protein ratio.

### Materials and Methods

Sample input to the Spectralys analysis was YghJ isolated from
i) wild type UPEC strain (yghJ-FLAG tag relying on chromosomal expression levels,
ii) the production strain (ΔwaaL, ΔfimH, ΔpapG, yghJ-GS linker-10×His tag) relying on chromosomal expression levels and
iii) artificially induced protein expression from plasmid in a standard E. coli K12 genetic background.

To determine the glycan/peptide ratio Fourier-transform Infrared spectroscopy was performed on the sample input to obtain FTIR spectra. Subsequently, the spectra have been integrated between 1182 and 1002 cm-1 (glycan absorption) and between 1740 and 1478 cm-1 (protein absorption) to obtain the peak area ratio which reflects the mass ratio between carbohydrates and proteins. For more detailed information, the method is described in Derenne et al. ((2021). Analysis of Glycoproteins by ATR-FTIR Spectroscopy: Comparative Assessment. In: Delobel, A. (eds) Mass Spectrometry of Glycoproteins. Methods in Molecular Biology, vol 2271. Humana, New York, NY. https://doi.org/10.1007/978-1-0716-1241-5_25).

TABLE 7
showing the genetic background of the
strains used for YghJ purification.

| Sample | Genetic background |
|---|---|
| Wild type UPEC | yghJ-FLAG-tag |
| Production strain | Δwaal, ΔfimH, ΔpapG, yghJ-GS linker-10xHis |
| Standard E. coli | K12 commensal E. coli |
| production strain | |

### Results

The results from the Spectralys analysis is shown in FIG. 11. The analysis shows that YghJ isolated from either the wild type UPEC strain or the production strain have identical glycan to protein ratios of 0.034. In contrast, plasmid expression in a conventional E. coli production strain resulted in the lowest levels of glycosylation with a ratio of only 0.018.

### Conclusion

the current industry standard for protein expression in conventional E. coli results in proteins dissimilar to what “nature” produces, when considering the glycan to protein ratio. This claim is supported by FIG. 1, which shows the YghJ protein isolated from a standard E. coli production strain resulted in a much lower number of unique glycopeptides. Further support for this claim is shown in FIG. 2 which demonstrates that the relative glycopeptide abundance and pattern was very dissimilar between the production strain according to the invention and the standard E. coli expression strain. This is again further supported by the data in example 7 and FIG. 11, which also shows that the glycan to protein varies between standard production strains and wt strains and the production strain according to the invention.

In sum, it the data indicates that current industry production standards result in low efficacy vaccines. On the other hand, YghJ isolated from the production strain which has an intact protein glycosylation apparatus results in a protein where the total glycan to protein content shows resemblance to the wild type UPEC strain.

### Sequences

SEQ ID NO: 1: (1520 AA)
MNKKFKYKKSLLAAILSATLLAGCDGGGSGSSSDTPSVDSGSGTLP
EVKPDPTPTPEPTPEPTPDPEPTPDPTPDPEPTPEPEPEPVPTKT
GYLTLGGSQRVTGATCNGESSDGFTFTPGNTVSCVVGSTTIATFN
TQSEAARSLRAVDKVSFSLEDAQELANSENKKTNAISLVTSSDSC
PADAEQLCLTFSSVVDRARFEKLYKQIDLATDNFSKLVNEEVENN
AATDKAPSTHTSTVVPVTTEGTKPDLNASFVSANAEQFYQYQPTE
IILSEGQLVDSLGNGVAGVDYYTNSGRGVTDENGKFSFSWGETIS
FGIDTFELGSVRGNKSTIALTELGDEVRGANIDQLIHRYSTTGQN
NTRVVPDDVRKVFAEYPNVINEIINLSLSNGATLDEGDQNVVLPN
EFIEQFKTGQAKEIDTAICAKTDGCNEARWFSLTTRNVNDGQIQG
VINKLWGVDTNYQSVSKFHVFHDSTNFYGSTGNARGQAVVNISNS
AFPILMARNDKNYWLAFGEKRAWDKNELAYITEAPSIVQPENVTR
DTATFNLPFISLGQVGEGKLMVIGNPHYNSILRCPNGYSWGGGVN
SKGECTLSGDSDDMKHFMQNVLRYLSNDIWQPNTKSIMTVGTNLE
NVYFKKAGQVLGNSAPFAFHEDFTGITVKQLTSYGDLNPEEIPLL
ILNGFEYVTQWSGDPYAVPLRADTSKPKLTQQDVTDLIAYLNKGG
SVLIMENVMSNLKEESASSFVRLLDAAGLSMALNKSVVNNDPQGY
PDRVRQRRATGIWVYERYPAADGAQPPYTIDPNTGEVTWKYQQDN
KPDDKPKLEVASWQEEVEGKQVTRYAFIDEAEYTTEESLEAAKAK
IFEKFPGLQECKDSTYHYEINCLERRPGTDVPVTGGMYVPRYTQL
NLDADTAKAMVQAADLGTNIQRLYQHELYFRTKGSKGERLNSVDL
ERLYONMSVWLWNDTKYRYEEGKEDELGFKTFTEFLNCYANDAYA
GGTKCSADLKKSLVDNNMIYGDGSSKAGMMNPSYPLNYMEKPLTR
LMLGRSWWDLNIKVDVEKYPGSVSAKGESVTENISLYSNPTKWFA
GNMQSTGLWAPAQQDVTIKSSASVPVTVTVALADDLTGREKHEVA
LNRPPRVTKTYTLEANGEVTFKVPYGGLIYIKGDSKDDVSANFTF
TGVVKAPFYKDGEWKNDLDSPAPLGELESASFVYTTPKKNLEASN
FTGGVAEFAKDLDTFASSMNDFYGRNDEDGKHRMFTYKNLTGHKH
RFTNDVQISIGDAHSGYPVMNSSFSTNSTTLPTTPLNDWLIWHEV
GHNAAETPLNVPGATEVANNVLALYMQDRYLGKMNRVADDITVAP
EYLDESNGQAWARGGAGDRLLMYAQLKEWAEENFDIKOWYPDGEL
PKFYSDRKGMKGWNLFQLMHRKARGDDVGNSTFGGKNYCAESNGN
AADTLMLCASWVAQADLSEFFKKWNPGASAYQLPGATEMSFQGGV
SSSAYSTLASLKLPKPEKGPETINKVTEHKMSAE

### REFERENCES

Alam, M. M., Arifuzzaman, M., Ahmad, S. M., Hosen, M. I., Rahman, M. A., Rashu, R., et al. (2013). Study of avidity of antigen-specific antibody as a means of understanding development of long-term immunological memory after Vibrio cholerae 01 infection. Clin. Vaccine Immunol. 20, 17-23. doi: 10.1128/CVI.00521-12.
Boysen, A., Palmisano, G., Krogh, T. J., Duggin, I. G., Larsen, M. R., Møller-Jensen, J., et al. (2016). A novel mass spectrometric strategy “BEMAP” reveals Extensive O-linked protein glycosylation in Enterotoxigenic Escherichia coli. Sci. Rep. 6, 32016. doi: 10.1038/srep32016.
Cassini, A., Plachouras, D., Eckmanns, T., Abu Sin, M., Blank, H. P., Ducomble, T., et al. (2016). Burden of Six Healthcare-Associated Infections on European Population Health: Estimating Incidence-Based Disability-Adjusted Life Years through a Population Prevalence-Based Modelling Study. PLoS Med. 13, 1-16. doi: 10.1371/journal.pmed.1002150.
Cek, M., Tandoǧdu, Z., Wagenlehner, F., Tenke, P., Naber, K., and Bjerklund-Johansen, T. E. (2014). Healthcare-associated urinary tract infections in hospitalized urological patients—a global perspective: results from the GPIU studies 2003-2010. World J. Urol. 32, 1587-1594. doi: 10.1007/s00345-013-1218-9.
Datsenko, K. A., and Wanner, B. L. (2000). One-step inactivation of chromosomal genes in Escherichia coli K-12 using PCR products. Proc. Natl. Acad. Sci. U.S.A. 97, 6640-6645. doi: 10.1073/pnas.120163297.
Hilgers, L. A. T., Platenburg, P. P. L. I., Bajramovic, J., Veth, J., Sauerwein, R., Roeffen, W., et al. (2017). Carbohydrate fatty acid monosulphate esters are safe and effective adjuvants for humoral responses. Vaccine 35, 3249-3255. doi: 10.1016/j.vaccine.2017.04.055.
Luo, Q., Vickers, T. J., and Fleckenstein, J. M. (2016). Immunogenicity and protective efficacy against enterotoxigenic Escherichia coli colonization following intradermal, sublingual, or oral vaccination with EtpA adhesin. Clin. Vaccine Immunol. 23, 628-637. doi: 10.1128/CVI.00248-16.
Medina, M., and Castillo-Pino, E. (2019). An introduction to the epidemiology and burden of urinary tract infections. Ther. Adv. Urol. 11, 3-7.
Mitchell, B. G., Ferguson, J. K., Anderson, M., Sear, J., and Barnett, A. (2016). Length of stay and mortality associated with healthcare-associated urinary tract infections: A multi-state model. J. Hosp. Infect. 93, 92-99. doi: 10.1016/j.jhin.2016.01.012.
Nesta, B., Valeri, M., Spagnuolo, A., Rosini, R., Mora, M., Donato, P., et al. (2014). SsIE Elicits Functional Antibodies That Impair In Vitro Mucinase Activity and In Vivo Colonization by Both Intestinal and Extraintestinal Escherichia coli Strains. PLoS Pathog. 10. doi: 10.1371/journal.ppat.1004124.
Nielsen, T. K., Petersen, N. A., Stærk, K., Grønnemose, R. B., Palarasah, Y., Nielsen, L. F., et al. (2019). A Porcine Model for Urinary Tract Infection. Front. Microbiol. 10, 1-12. doi: 10.3389/fmicb.2019.02564.
Riaz, S., Steinsland, H., Thorsing, M., Andersen, A. Z., Boysen, A., and Hanevik, K. (2021). Characterization of Glycosylation-Specific Systemic and Mucosal IgA Antibody Responses to Escherichia coli Mucinase YghJ (SsIE). Front. Immunol. 12, 1-11. doi: 10.3389/fimmu.2021.760135.
Stærk, K., Andersen, M. Ø, and Andersen, T. E. (2022). Uropathogenic Escherichia coli can cause cystitis at extremely low inocula in a pig model. J. Med. Microbiol. 71, 1-5. doi: 10.1099/jmm.0.001537.
The European Centre for Disease Prevention and Control (2017). ECDC: SURVEILLANCE REPORT. Surveillance of antimicrobial resistance in Europe 2016. doi: 10.2900/296939.
Thorsing, M., Krogh, T. J., Vitved, L., Nawrocki, A., Jakobsen, R., Larsen, M. R., et al. (2021). Linking inherent O-Linked Protein Glycosylation of YghJ to Increased Antigen Potential. Front. Cell. Infect. Microbiol. 11, 1-10. doi: 10.3389/fcimb.2021.705468.
Vallejo-Torres, L., Pujol, M., Shaw, E., Wiegand, I., Vigo, J. M., Stoddart, M., et al. (2018). Cost of hospitalised patients due to complicated urinary tract infections: A retrospective observational study in countries with high prevalence of multidrug-resistant Gram-negative bacteria: The COMBACTE-MAGNET, RESCUING study. BMJ Open 8, 1-9. doi: 10.1136/bmjopen-2017-020251.
Wang, X., and Quinn, P. J. (2010). Lipopolysaccharide: Biosynthetic pathway and structure modification. Prog. Lipid Res. 49, 97-107. doi: 10.1016/j.plipres.2009.06.002.
Zhao, D., Yuan, S., Xiong, B., Sun, H., Ye, L., Li, J., et al. (2016). Development of a fast and easy method for Escherichia coli genome editing with CRISPR/Cas9. Microb. Cell Fact. 15, 1-9. doi: 10.1186/s12934-016-0605-5.
Zhao, X., Wang, L., Wei, N., Zhang, J., Ma, W., Zhao, H., et al. (2020). Epidemiological and clinical characteristics of healthcare-associated infection in elderly patients in a large Chinese tertiary hospital: A 3-year surveillance study. BMC Infect. Dis. 20, 1-7. doi: 10.1186/s12879-020-4840-3.

1. A genetically modified E. coli which:
does not express FimH; does not express PapG; and
does not express WaaL.

2. The genetically modified E. coli according to claim 1, being an extra-intestinal pathogenic E. coli (ExPEC).

3. The genetically modified E. coli according to claim 1, being UTI89.

4. The genetically modified E. coli according to claim 1, wherein said genetically modified E. coli express a YGHJ glycosylated polypeptide.

5. The genetically modified E. coli according to claim 1, being avirulent and/or non-pathogenic and/or not capable of causing disease.

6. (canceled)

7. The genetically modified E. coli according to claim 1, wherein said genetically modified E. coli
is adapted to express a YGHJ glycosylated polypeptide.

8. The genetically modified E. coli according to claim 1, encoding for a YGHJ polypeptide which can be expressed in said E. coli, with a glycosylation pattern similar to a non-genetically modified version of said E. coli UTI89.

9. The genetically modified E. coli according to claim 1, wherein the genetically modified E. coli has lower adherence to human bladder cells, than E. coli K-12 MG1655.

10. The genetically modified E. coli according to claim 1, encoding for a YGHJ polypeptide wherein said polypeptide is expressed from the genome.

11. The genetically modified E. coli according to claim 1, expressing an ExPEC-derived polypeptide comprising:
a) an amino acid sequence according to SEQ ID NO: 1; and/or
b) an amino acid sequence having at least 90% sequence identity to SEQ ID NO: 1; and/or
c) an amino acid sequence which is a fragment of at least 1000 consecutive amino acids from SEQ ID NO: 1; and/or
d) an amino acid sequence having at least 90% sequence identity to SEQ ID NO: 1 and including a fragment of at least 1000 consecutive amino acids from SEQ ID NO: 1.

12. The genetically modified E. coli according to claim 1, encoding for a YGHJ polypeptide, wherein the polypeptide is expressed from an exogenous vector.

13. The genetically modified E. coli according to claim 1, encoding for a YGHJ polypeptide, wherein the polypeptide is expressed from a plasmid.

14. The genetically modified E. coli according claim 1, expressing an ExPEC-derived polypeptide comprising:
a) an amino acid sequence according to SEQ ID NO: 1; and/or
b) an amino acid sequence having at least 90% sequence identity to SEQ ID NO: 1.

15. The genetically modified E. coli according to claim 1, expressing an amino acid sequence according to SEQ ID NO: 1.

16. (canceled)

17. The genetically modified E. coli according to claim 1 which:
does not express FimH;
does not express PapG; and
does not express WaaL;
wherein the genetically modified E. coli is UTI89:
wherein the genetically modified E coli, expresses:
a) an amino acid sequence according to SEQ ID NO: 1; and/or
b) an amino acid sequence having at least 90% sequence identity to SEQ ID NO: 1; and/or
c) an amino acid sequence which is a fragment of at least 1000 consecutive amino acids from SEQ ID NO: 1; and/or
d) an amino acid sequence having at least 90% sequence identity to SEQ ID NO: 1 and including a fragment of at least 1000 consecutive amino acids from SEQ ID NO: 1;
wherein said polypeptide has a glycosylation pattern defined by being glycosylated on at least 15 positions in SEQ ID NO: 1, selected from the group depicted in Table 2A-B.

18. The genetically modified E. coli according to claim 1, which
does not express FimH;
does not express PapG; and
does not express WaaL:
wherein the genetically modified E. coli is UTI89;
wherein the genetically modified E. coli, expresses an YGHJ polypeptide.

19. The genetically modified E. coli according to claim 1 which:
does not express FimH;
does not express PapG; and
does not express WaaL;
wherein the genetically modified E. coli is UTI89; and
wherein the genetically modified E. coli, expresses an amino acid sequence according to SEQ ID NO: 1.

20.-36. (canceled)
